(19) United States
(12) Patent Application Publication
(10) Pub. No.: US 2020/0029939 A1
(43) Pub. Date: Jan. 30, 2020

(54) OPERATION METHOD OF ULTRASOUND OBSERVATION APPARATUS, AND ULTRASOUND OBSERVATION APPARATUS

(71) Applicant: OLYMPUS CORPORATION, Tokyo (JP)

(72) Inventor: Tomonao KAWASHIMA, Tokyo (JP)

(73) Assignee: OLYMPUS CORPORATION, Tokyo (JP)

(21) Appl. No.: 16/590,927

(22) Filed: Oct. 2, 2019

Related U.S. Application Data
(63) Continuation of application No. PCT/JP2018/014621, filed on Apr. 5, 2018.

(30) Foreign Application Priority Data
Apr. 5, 2017 (JP) 2017-075330

(51) Int. Cl. A61B 8/08

(52) U.S. Cl. CPC A61B 8/5207; A61B 8/469; A61B 8/5269; A61B 8/5246

(57) ABSTRACT

An ultrasound observation apparatus is configured to be able to exchange plural models of ultrasound probes and configured to receive an ultrasound signal from an ultrasound probe connected to the ultrasound observation apparatus. The ultrasound observation apparatus includes: a corrector configured to correct ultrasound data based on the ultrasound signal using first reference data and second reference data, the first reference data being determined by different models of the ultrasound probes to be connected to the ultrasound observation apparatus, the second reference data being determined by individual probes of the same models of the ultrasound probes to be connected to the ultrasound observation apparatus. The corrector is configured to correct the ultrasound signal by performing a calculation on the ultrasound signal using each of the first reference data and the second reference data for each frequency or each distance.

### CROSS-REFERENCE TO RELATED APPLICATION

This application is a continuation of PCT International Application No. PCT/JP2018/014621 filed on Apr. 5, 2018, which designates the United States, incorporated herein by reference, and which claims the benefit of priority from Japanese Patent Application No. 2017-075330, filed on Apr. 5, 2017, incorporated herein by reference.

### BACKGROUND

### 1. Technical Field

The present disclosure relates to an operation method of an ultrasound observation apparatus, and an ultrasound observation apparatus for observing tissue to be observed using ultrasound.

### 2. Related Art

A technique to receive ultrasound echoes that are backscattered by a subject to be observed with an ultrasound transducer, transform the ultrasound echoes into an ultrasound signal, calculate feature data from frequency spectrum of the resultant ultrasound signal, and image the calculated feature data is known as a technique to represent tissue characterization of a subject to be observed using ultrasound (for example, refer to Japanese Patent No. 3981366). The acoustic wave scattering is a physical phenomenon that acoustic waves collide with particles in a medium, the acoustic waves and the particles exert force to each other (referred to as interaction), and this enables the acoustic waves to change their travel direction. Furthermore, backscattering refers to components that return in a direction of a sound source in scattering. The phenomenon is generally referred to as reflection but the term of backscattering will be used below herein. The sound source is an ultrasound transducer. In the technique, feature data of a frequency spectrum is extracted as an analysis value representing tissue characterization of a subject to be observed. Thereafter, a feature data image to which visual information corresponding to the feature data, such as color information, is assigned is generated. The feature data image is then superimposed onto an ultrasound image based on an ultrasound signal to generate a superimposed image and the superimposed image is displayed. A technologist, such as a doctor, is able to diagnose the tissue characterization of the subject to be observed by looking at the superimposed image that is displayed.

To give accurate diagnosis using a feature data image, it is important to perform signal processing according to the properties of an ultrasound probe including an ultrasound transducer. For example, Japanese Patent No. 3981366 discloses a technique to correct an ultrasound signal according to the degree of degradation of the ultrasound probe. According to Japanese Patent No. 3981366, even when the ultrasound probe degrades, it is possible to reduce degradation of an ultrasound image by performing correction to approximate the signal intensity after degradation to the signal intensity before degradation on an acquired signal.

### SUMMARY

In some embodiments, provided is an operation method of an ultrasound observation apparatus configured to be able to exchange plural models of ultrasound probes and configured to receive an ultrasound signal from an ultrasound probe connected to the ultrasound observation apparatus. The method includes: correcting ultrasound data based on the ultrasound signal using first reference data and second reference data, the first reference data being determined by different models of the ultrasound probes to be connected to the ultrasound observation apparatus, the second reference data being determined by individual probes of the same models of the ultrasound probes to be connected to the ultrasound observation apparatus. The correcting includes correcting the ultrasound signal by performing a calculation on the ultrasound signal using each of the first reference data and the second reference data for each frequency or each distance.

In some embodiments, provided is an ultrasound observation apparatus configured to be able to exchange plural models of ultrasound probes and configured to receive an ultrasound signal from an ultrasound probe connected to the ultrasound observation apparatus. The ultrasound observation apparatus includes: a corrector configured to correct ultrasound data based on the ultrasound signal using first reference data and second reference data, the first reference data being determined by different models of the ultrasound probes to be connected to the ultrasound observation apparatus, the second reference data being determined by individual probes of the same models of the ultrasound probes to be connected to the ultrasound observation apparatus. The corrector is configured to correct the ultrasound signal by performing a calculation on the ultrasound signal using each of the first reference data and the second reference data for each frequency or each distance.

### BRIEF DESCRIPTION OF THE DRAWINGS

FIG. 1 is a block diagram illustrating a configuration of an ultrasound diagnostic system including an ultrasound observation apparatus according to a first embodiment of the present disclosure;

FIG. 2 is a graph representing a relationship between reception depth and amplification factor in an amplification process that is performed by a transmitter-receiver;

FIG. 3 is a diagram schematically illustrating an area that is scanned by an ultrasound transducer and sound ray data;

FIG. 4 is a diagram schematically illustrating a data array in RF data on one sound ray of an ultrasound signal;

FIG. 5 is a conceptual diagram illustrating a difference in effect on subject spectral data resulting from an individual difference of an ultrasound endoscope and a model difference of the ultrasound observation apparatus;

FIG. 6 is a diagram illustrating spectral data that is acquired in advance;

FIG. 7 is a diagram illustrating spectral data that is acquired in advance;

FIG. 8 is a graph representing exemplary spectral data that is calculated by a spectral corrector of the ultrasound observation apparatus according to the first embodiment of the disclosure;

FIG. 9 is a graph representing a straight line having, as a parameter, post-correction feature data that is calculated by a normal feature data calculator of the ultrasound observation apparatus according to the first embodiment of the disclosure;

FIG. 10 is a flowchart illustrating an overview of a process that is performed by the ultrasound observation apparatus according to the first embodiment of the disclosure;

FIG. 11 is a diagram illustrating a screen to choose model information on the ultrasound endoscope and the ultrasound observation apparatus;

FIG. 12 is a diagram illustrating a screen to choose individual information on ultrasound endoscopes;

FIG. 13 is a flowchart illustrating an overview of a process that is executed by a frequency analyzer of the ultrasound observation apparatus according to the first embodiment of the disclosure;

FIG. 14 is a diagram schematically illustrating exemplary display of a synthesized image on a display device of the ultrasound observation apparatus according to the first embodiment of the disclosure;

FIG. 15 is a diagram illustrating acquisition of reference spectral data performed by the ultrasound observation apparatus;

FIG. 16 is a conceptual view illustrating a difference in effect on subject spectral data resulting from an individual difference of the ultrasound endoscope and an individual difference of the ultrasound observation apparatus;

FIG. 17 is a block diagram illustrating a configuration of an ultrasound diagnostic system including an ultrasound observation apparatus according to a second embodiment of the disclosure;

FIG. 18 is a flowchart illustrating an overview of a process performed by the ultrasound observation apparatus according to the second embodiment of the present disclosure;

FIG. 19 is a block diagram illustrating a configuration of an ultrasound diagnostic system including an ultrasound observation apparatus according to a third embodiment of the disclosure;

FIG. 20 is a block diagram illustrating a configuration of an ultrasound diagnostic system including an ultrasound observation apparatus according to a fourth embodiment of the disclosure; and

FIG. 21 is a block diagram illustrating a configuration of an ultrasound diagnostic system including an ultrasound observation apparatus according to a fifth embodiment of the disclosure.

### DETAILED DESCRIPTION

Embodiments for carrying out the present disclosure (“embodiment” below) will be described with reference to the accompanying drawings.

### First Embodiment

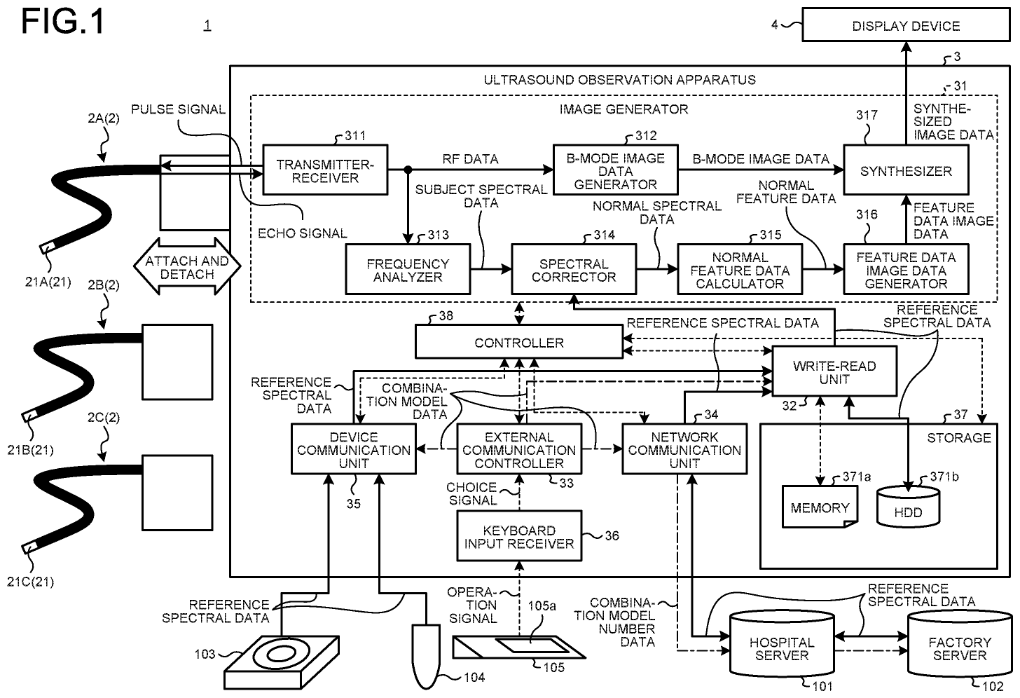

FIG. 1 is a block diagram of a configuration of an ultrasound diagnostic system 1 including an ultrasound observation apparatus 3 according to a first embodiment of the present disclosure. The ultrasound diagnostic system 1 illustrated in FIG. 1 includes an ultrasound endoscope 2 (ultrasound endoscopes 2A to 2C) that transmits ultrasound to an observation subject to be observed and receives the ultrasound that is backscattered by the observation subject; the ultrasound observation apparatus 3 that generates an ultrasound image based on an ultrasound signal that is acquired by the ultrasound endoscope 2 that is connected to the ultrasound observation apparatus 3; and a display device 4 that displays the ultrasound image that is generated by the ultrasound observation apparatus 3. Any one of the ultrasound endoscopes 2A to 2C is detachably connectable to the ultrasound observation apparatus 3. In the first embodiment, the ultrasound endoscope 2 functions as an ultrasound probe. In the block diagram described below, the solid arrows represent transmission of electric signals, spectral data and feature data, the arrows in dashed-dotted lines represent transmission of combination model number data, and the arrows in dotted lines represent transmission of electric signals and data relating to control, etc.

The ultrasound endoscope 2A includes an ultrasound transducer 21A on it distal end part. The ultrasound transducer 21A converts an electric pulse signal that is received from the ultrasound observation apparatus 3 into ultrasound pulses (acoustic pulses) and applies the ultrasound pulses to the observation subject and converts ultrasound echoes that are backscattered by the observation subject into an electric echo signal expressing the ultrasound echoes by a change in voltage. The ultrasound endoscopes 2B and 2C similarly include ultrasound transducers 21B and 21C, respectively.

The ultrasound endoscopes 2A to 2C will be described as ultrasound endoscopes where the ultrasound transducers 21A to 21C are of models different from one another. As for each of the models of the ultrasound endoscopes 2A to 2C, there are other ultrasound endoscopes whose individual numbers are different from one another. For example, assuming that the model of the ultrasound endoscope 2A is P, there are multiple ultrasound endoscopes 2A of the model P having different individual numbers. Similarly, assuming that the model of the ultrasound endoscope 2B is Q and the model of the ultrasound endoscope 2C is R, there are multiple ultrasound endoscopes 2B of the model Q having different individual numbers and multiple ultrasound endoscopes 2C of the model R having different individual numbers.

Each of the ultrasound endoscopes 2A to 2C includes an elongated insertion unit to be inserted into the observation subject. The insertion unit generally includes an imaging optical system and an imaging device on its distal end part and, when the observation subject is a subject in a human body, the insertion unit is inserted into the corresponding digestive tract (the esophagus, the stomach, the duodenum or the large intestine) or the corresponding respiratory organ (the trachea or a bronchi), making it possible to capture images of the digestive tract or the respiratory organ and their surrounding organs (the pancreas, the gallbladder, the bile duct, the bile tract, lymph nodes, mediastinal organs, blood vessels, etc.). In the first embodiment, the observation subject in the case where tissue in the human body, or the like, is observed in a facility, such as a hospital, will be particularly referred to as a subject. The insertion unit generally incorporates an elongated light guide that guides the illumination light that is applied to the observation subject to capture an image. While the distal end part of the light guide reaches the distal end of the insertion unit, the proximal end of the light guide is connected to the light source device that generates illumination light.

The ultrasound observation apparatus 3 includes an image generator 31 that generates image data based on the echo signal that is acquired from the ultrasound endoscope; a write-read unit 32 that writes and reads reference spectral data for the image generator 31 to generate image data; an external communication controller 33 that controls communication with an external unit to acquire the reference spectral data; a network communication unit 34 that acquires the reference spectral data via a communication network that is achieved with, for example, an existing public network, a local area network (LAN) or a wired area network (WAN); and a device communication unit 35 that communicates with a devices that is connected to the ultrasound observation apparatus 3; a keyboard input receiver 36 that receives input from a keyboard; a storage 37 that stores various types of information necessary for operations of the ultrasound observation apparatus 3; and a controller 38 that overall controls the ultrasound diagnostic system 1.

The image generator 31 includes a transmitter-receiver 311 that is electrically connected to the ultrasound endoscope 2, transmits a transmission signal (the pulse signal) consisting of a high-voltage pulse to an ultrasound transducer 21 based on a given waveform and given timing, receives the echo signal that is an electric radio frequency (RF) signal from the ultrasound transducer 21, generates digital data (RF data below) by performing A/D conversion described below on the echo signal, and outputs the RF data; a B-mode image data generator 312 that generates B-mode image data based on the RF data that is received from the transmitter-receiver 311; a frequency analyzer 313 that calculates subject spectral data by performing fast Fourier transform (FFT) on the RF data that is generated by the transmitter-receiver 311 and performing frequency analysis; a spectral corrector 314 that performs correction corresponding to the model and individual of the ultrasound endoscope 2 and the model of the ultrasound observation apparatus 3 on the subject spectral data that is calculated by the frequency analyzer 313; a normal feature data calculator 315 that calculates normal feature data based on the normal spectral data that is generated by the spectral corrector 314; a feature data image data generator 316 that assigns color information according to the normal feature data, which is calculated by the normal feature data calculator 315, to generate feature data image data; and a synthesizer 317 that generates synthesized image data by synthesizing the feature data image data, which is generated by the feature data image data generator 316, with the B-mode image data, which is generated by the B-mode image data generator 312.

The function of each unit in the image generator 31 in the ultrasound observation apparatus 3 will be described.

The transmitter-receiver 311 amplifies the received echo signal.

After performing processing, such as filtering, on the amplified echo signal, the transmitter-receiver 311 performs sampling at an appropriate sampling frequency (for example, 50 MHz) for discretization (that is, A/D conversion). Accordingly, the transmitter-receiver 311 generates RF data obtained by performing discretization on the amplified echo signal and outputs the RF data to the B-mode image data generator 312 and the frequency analyzer 313. When the ultrasound endoscope 2 has a configuration to cause the ultrasound transducer 21 including a plurality of devices that are provided in an array to perform electric scanning, the transmitter-receiver 311 includes a multichannel circuit for beam synthesis corresponding to the devices.

For the frequency band of the pulse signal transmitted by the transmitter-receiver 311, a wide band covering almost all a liner response frequency band of the ultrasound transducer 21 for electroacoustic conversion from the pulse signal to an ultrasound pulse performed by the ultrasound transducer is set. For various processing frequency bands of echo signals in the transmitter-receiver 311, a wide band covering almost all a liner response frequency band of the ultrasound transducer 21 for the ultrasound transducer 21 to perform electroacoustic conversion from ultrasound echoes into an ultrasound pulse. This enables accurate approximation when approximation on a frequency spectrum to be described below is executed.

The transmitter-receiver 311 may be provided with a function of transmitting various control signals that are output by the controller 38 to the ultrasound endoscope 2, of receiving various types of information containing an identifying ID (for example, model information) from the ultrasound endoscope 2, and of transmitting the information to the controller 38.

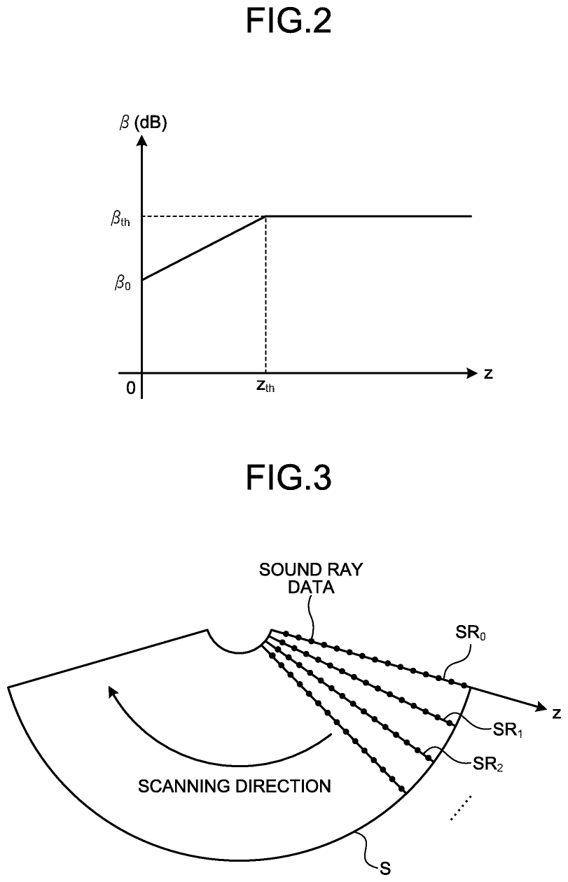

The B-mode image data generator 312 performs sensitivity time control (STC) correction in which, the greater the reception depth is, the higher the amplification rate at which the RF data is amplified is. FIG. 2 is a graph representing a relationship between reception depth and amplification factor in amplification that is performed by the transmitter-receiver 311. FIG. 2 is a logarithmic plot where the horizontal axis is taken as the reception depth and the vertical axis is taken as the common logarithm of the amplification factor β. The unit of the vertical axis is decibel (dB). The reception depth z represented in FIG. 2 is an amount that is calculated based on the elapse of time from the time point at which reception of ultrasound is started. On the logarithmic plot represented in FIG. 2, when the reception depth z is under a threshold zₜₕ, the amplification factor β increases linearly from β₀ to Bₜₕ (>β₀) with an increase of the reception depth z. When the reception depth z is at or above the threshold zₜₕ, the amplification factor β takes a constant value βₜₕ. The value of the threshold zₜₕ is a value at which the ultrasound signal that is received from the observation subject almost attenuates and thus noise is dominant. The relationship represented in FIG. 2 is stored in the storage 37 in advance.

Furthermore, the B-mode image data generator 312 applies a band-pass filter to the RF data and performs envelope detection on the RF data to generate data representing the amplitude or intensity of the echo signal. The B-mode image data generator 312 then performs known processing, such as logarithmic transformation, on the data to generate digital sound ray data. In logarithmic transformation, the data representing the amplitude or intensity of the echo signal is divided by a specific voltage V_{c} referred to as a reference voltage (reference voltage V_{c} below) and a common logarithm thereof is taken to transform the data. The transformed data is expressed in a decibel value. The sound ray data is data in which values proportional to the digits representing the amplitude or intensity of the echo signal representing the intensity of backscattering of ultrasound pulses by decimal are arranged along the direction in which the ultrasound pulses are transmitted and received (the depth direction).

FIG. 3 is a diagram schematically illustrating an area that is scanned by the ultrasound transducer 21 (that can be simply referred to as the scanned area) and sound ray data. The scanned area S illustrated in FIG. 3 forms a sector. FIG. 3 expresses the routes in which ultrasound travels back and forth linearly and expresses sound ray data by dots that are arranged on each of the sound rays. For convenience of the following description, the sound rays are denoted with numbers 1, 2, 3 . . . , respectively, from the start of scanning (on the right in FIG. 3) sequentially and the first sound ray is defined as SR₁, the second sound ray is defined as SR₂, the third sound ray is defined as SR₃, . . . , and the k-th sound ray is defined as SRₖ. FIG. 3 corresponds to the case where the ultrasound transducer 21 is a convex transducer. FIG. 3 represents the reception depth of the sound ray data by z. When the ultrasound pulses that are applied from the surface of the ultrasound transducer 21 backscatter in the object at the reception depth z and return as ultrasound echoes to the ultrasound transducer 21, a round-trip distance L and the reception depth z has a relationship z=L/2.

The B-mode image data generator 312 performs signal processing using known techniques, such as gain processing and contrast processing, on the sound ray data.

After coordinate transformation in which the sound day data is rearranged is preformed such that the generated sound-ray data correctly expresses the scanned area spatially, the B-mode image data generator 312 buries the gaps between sets of sound ray data by performing interpolation between the sets of sound ray data, thereby generating B-mode image data. A B-mode image is a grayscale image where the R (red), G (green), and B (blue) values that are variables in the case where the RGB coordinate system is employed as a color space are caused to agree with one another. The B-mode image data generator 312 outputs the generated B-mode image data to the synthesizer 317. The B-mode image data generator 312 is achieved using a dedicated integrated circuit that implements a specific function of a general-purpose processor like a central processing unit (CPU), an application specific integrated circuit (ASIC), a field programmable gate array (ASIC), or the like.

The frequency analyzer 313 sections the RF data (line data) on each sound ray that is generated by the transmitter-receiver 311 into multiple blocks at given relatively short time intervals and performs FFT on the RF data on each of the sectioned blocks (“RF data string” below), thereby calculating a frequency spectrum of each block of the sound ray. The “frequency spectrum” herein means a “frequency distribution of intensity or voltage amplitude of the echo signal that is obtained from a certain reception depth z (that is, a certain round-trip distance L)” obtained by performing FFT on the RF data string. The “intensity” herein refers to any one of the voltage amplitude of the echo signal and the power of the echo signal.

For the first embodiment, the case where the frequency distribution of voltage amplitude of the echo signal is employed as the frequency spectrum will be described. The frequency analyzer 313 will be described as an example in the case where data of the frequency spectrum (also referred to as spectral data below) is generated based on a frequency component V(f, L) of voltage amplitude, where f is a frequency. After dividing the frequency component V(f, L) of the amplitude of the RF data (practically, the voltage amplitude of the echo signal) by the reference voltage V_{c} and performing logarithmic transformation in which the common logarithm (log) is taken and expressed by the decibel unit, the frequency analyzer 313 multiplies the common logarithm by an appropriate positive constant α, thereby generating spectral data S(f, L) on the observation subject that is given by Equation (1) below.

S(f, L)=α·log {v(f, L)/V_{c}} (1)

A method of calculating a frequency component V(f, L) of the voltage amplitude by frequency analysis performed by the frequency analyzer 313 will be specifically described below. In general, when the observation subject is a subject, such as human tissue, the frequency spectrum of the echo signal represent different tendency depending on the human tissue characterization that is scanned with ultrasound. This is because the frequency spectrum has a correlation with the size, the number density and the acoustic impedance of the scatterers that scatter ultrasound, etc. The human tissue characterization” herein refers to, for example, characteristics of tissue, such as a malignant tumor (cancer), a benign tumor, an endocrine tumor, a mucinous tumor, normal tissue, a cyst, or a vascular channel.

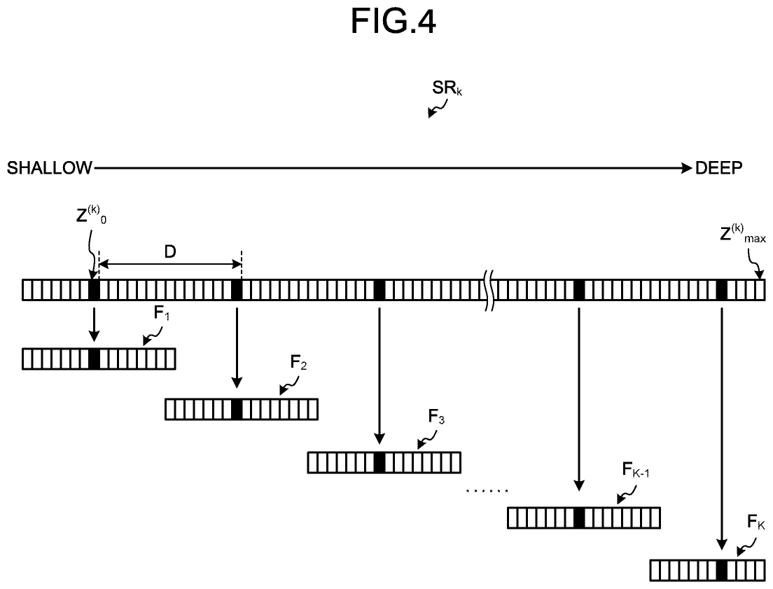

FIG. 4 is a diagram schematically illustrating a data array in the RF data on one sound ray SRₖ of the ultrasound signal. A white or black rectangle in the sound ray SRₖ means data at a sample point. In the RF data on the sound ray SRₖ, the more the data is positioned rightward, the deeper the area from which the RF data comes in the case where measurement is performed along the sound ray SRₖ from the ultrasound transducer 21 is (refer to the arrow in FIG. 4). As described above, the RF data on the sound ray SRₖ is RF data that is sampled from the echo signal by the A/D conversion at the transmitter-receiver 311 and is discritized. FIG. 4 illustrates the case where the eighth data position of the RF data on the sound ray SRₖ of the number k is set as an initial value Z^{(k)}₀ in the direction of the reception depth z, and any positon may be set for the initial value. The result of calculation by the frequency analyzer 313 is obtained in a complex number and is stored in the storage 37.

RF data strings Fⱼ (j=1,2, . . . , K) are blocks on which the FFT is to be performed among the RF data. In general, in order to perform FFT, the RF data has to include the number of sets of data corresponding to the power of 2. In this sense, data strings Fⱼ (j=1,2, . . . ,K−1) excluding Fₖ are normal RF data strings in each of which the number of sets of data is 16 (=2⁴). On the other hand, the RF data string Fₖ is an anomalous RF data string because the number of sets of data is 12. When FFT is performed on the anomalous RF data string, by inserting zero data by the volume of deficiency, processing to generate a normal RF data string is performed. This aspect will be described in detail when the process performed by the frequency analyzer 313 is described (see FIG. 4). Thereafter, as described above, the frequency analyzer 313 executes FFT, executes a frequency component V(f, L) of voltage amplitude and, according to Equation (1) described above, calculate subject spectral data S(f, L). A frequency analyzer 313 further repeats the operation on all the sound rays illustrated in FIG. 3 to calculate spectral data S(f, L) omni-directionally and outputs the spectral data S(f, L) to the spectral corrector 314 (hereinafter, the “orientation” will be described as directions in which sets of sound ray data are oriented respectively over the scanning direction in FIG. 3).

The spectral corrector 314 calculates normal spectral data S_{c}(f, L) by correcting the subject spectral data S(f, L) that is output from the frequency analyzer 313. In the following description, for example, subject spectral data that is subject spectral data obtained by capturing an image of a human body, particularly, a living body (LB) using the ultrasound endoscope 2 and that is subject spectral data in the case where the parameters are the frequency f and the reception depth z is notated by S(LB; f, z). Similarly, reference spectral data that is obtained by capturing an image of a reference specimen by a combination of an ultrasound endoscope (Pᵢ) of Model P and an ultrasound observation apparatus (Bₘ) of Model B is notated by S(PᵢBₘ; f, z), where i and m are natural numbers representing individuals of the same model whose individual numbers are different from each other. What notated by the subscript of 0 represents a reference individual of the model.

As represented by Equation (2) below, the spectral corrector 314 calculates normal spectral data S_{c}(LB; f, z) by subtracting the reference spectral data S(PᵢBₘ; f, z) obtained by imaging the reference specimen from the subject spectral data S(LB; f, z) obtained by imaging the living body.

S_{c}(LB; f, z)=S(LB; f, z)−S(PᵢBₘ; f, z) (2)

Acquiring reference spectral data S(PᵢBₘ; f, z) obtained with all individuals of models on the market is extremely time-consuming and this results in an enormous volume of data. For this reason, in the first embodiment, utilizing the fact that Equation (3-1) and Equation (3-2) below hold, the spectral corrector 314 utilizes the right side of Equation (3-1) or (3-2) instead of the reference spectral data S(PᵢBₘ; f, z). The reason why Equation (3-1) and Equation (3-2) hold will be described below. Definitions of a model difference correction term ΔS₁₀ and an individual difference correction term ΔS₂₀ of Equation (3-2) will be also described below.

S(PᵢBₘ; f, z)=S(P₀B₀; f, z)−S(P₀A₀; f, z)+S(PᵢA₀; f, z) (3-1)

S(PᵢBₘ; f, z)=S(P₀A₀; f, z)+ΔS₁₀+ΔS₂₀ (3-2)

It is possible to take the first term and the second term of Equation (3-1) as model difference correction terms and the third term is an individual difference correction term or the first term is a model difference correction term and the second and third terms are individual difference correction terms. The model difference correction terms correspond to first reference data for model difference correction and the individual difference correction terms correspond to second reference data for individual difference correction.

In the first embodiment, from the standpoint of magnitude of effect on the subject spectral data, the model difference and individual difference of each of the ultrasound observation apparatus 3 and the ultrasound endoscope 2 should be discussed. The model difference affecting the subject spectral data is a difference resulting from a difference in design and the individual difference is a difference resulting from variation.

As for the ultrasound endoscope 2, as a cause of effect on the subject spectral data, a sensitivity difference of the ultrasound transducer 21 and its frequency characteristic difference and frequency characteristic difference of wiring, such as cables incorporated in the insertion unit of the ultrasound endoscope 2, are exemplified. It is considered that, among them, the sensitivity difference and frequency characteristic difference of sensitivity have great effects. In general, circuit designing and physical designing of size/material, etc., in the ultrasound endoscope 2 having effects thereon need not be uniform between models and thus efforts for uniformity are not made and accordingly the physical designing significantly differs between models. Thus, difference in designing may very well have an effect on the subject spectral data.

On the other hand, variations in the above-described sensitivity and frequency characteristics have an effect even on the B-mode image obtained by simple processing and it is still a difficult objective to reduce the effect. Reducing the effect on the subject spectral data is also considered to be a difficult objective. Thus, effects of both the difference and variations in designing on the subject spectral data should not be ignored. Accordingly, in the first embodiment, the model difference and individual difference of the ultrasound endoscope will not be ignored and will be dealt with.

As for the ultrasound observation apparatus 3, as a cause of effect on the subject spectral data, a drive waveform difference (difference in drive waveform) and a frequency characteristic differece in amplification in various receiving circuits in the transmitter receiver 311 are exemplified. It is considered that, among them, the drive waveform difference has a great effect. In general, circuit designing in the ultrasound observation apparatus 3 having an effect thereon need not be uniform between models and thus efforts for uniformity are not particularly made and thus the circuit designing significantly differs between models. Thus, a difference in designing may very well have an effect on the subject spectral data.

On the other hand, variations in the above-described drive waveform and variations in the above-described frequency characteristics have very little effect compared to the difference in designing when shipping examination is performed through. Thus, in the first embodiment, unless otherwise noted, the individual difference of the ultrasound observation apparatus is ignored. For all i and m representing individual numbers, Equation (4) below holds.

S(PᵢBₘ; f, z)=S(PᵢB_{C}; f, z) (4)

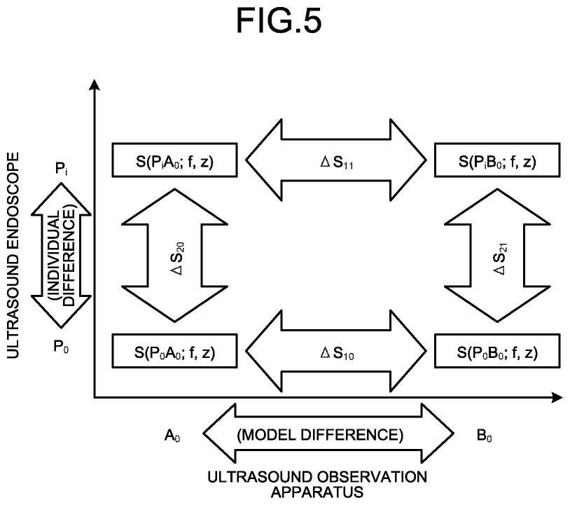

FIG. 5 is a conceptual diagram illustrating difference in effect on the subject spectral data resulting from the individual difference of ultrasound endoscopes and model difference of ultrasound observation apparatuses. The reason why FIG. 5 and Equation (3-1) and (3-2) hold will be described below.

The individual difference of the ultrasound observation apparatus 3 and the model difference between Model A and Model B will be looked at. Assume that a reference individual P₀ of Model P of the ultrasound endoscope 2 is connected to each of a reference individual A₀ of Model A and a reference individual B₀ of Model B and sets of reference spectral data S(P₀A₀; f, z) and S(P₀B₀; f, z) are obtained from a reference specimen. As described above, as a cause of effect on the subject spectral data, a drive waveform difference and a frequency characteristic difference in amplification in various receiving circuits in the transmitter receiver 311 are exemplified and the difference in designing appears as the model difference of the ultrasound observation apparatus 3. For example, V_{At}(f) is set for the frequency spectrum of the drive waveform in Model A and δ_{A}(f) is set for frequency characteristics of amplification. A frequency component V(f, L) of voltage amplitude serving as the basis of the reference spectral data S(P₀A₀; f, z) contains V_{At}(f) and δ_{A}(f) in a multiplication factor. As for the ultrasound observation apparatus 3, even when there is another factor of effect on the reference spectral data S(P₀A₀; f, z), the design value serving as the basis of the factor is generally contained as the multiplication factor of V(f, L).

According to Equation (1), calculation of S(P₀A₀; f, z) uses a common logarithm operation of V(f, L) and thus all these factors are contained as terms of addition forming S(P₀A₀; f, z). In other words, S(P_{C}A₀; f, z) contains a term of addition of α·log V_{At}(f)+α·log δ_{A}(f)+α·log (another factor). Eventually, as for the ultrasound observation apparatus 3, the design value serving as the basis of the cause of effect on the reference spectral data S(P₀A₀; f, z) is contained as the term of addition of S(P₀A₀; f, z) itself.

Similarly, for example, V_{Bt}(f) is set for each of frequency spectra of the drive waveform and δ_{B}(f) is set for the frequency characteristics of amplification. As for the ultrasound observation apparatus 3, the design value serving as the basis of the cause of effect on the reference spectral data S(P₀B₀; f, z) is contained as a term of addition. In other words, S(P₀B₀; f, z) contains a term of addition of α·log V_{Bt}(f)+α·log δ_{B}(f)+α·log (another factor).

A difference of ΔS₁₀ between both the sets of reference spectral data is defined by Equation (5-1) below.

ΔS₁₀=S(P₀B_{c}; f, z)−S(P₀A₀; f, z) (5-1)

The reference specimen and the ultrasound endoscope P₀ to be combined are common and thus common terms are offset in the process of subtraction of Equation (5-1) and the above-described difference in designing is obtained. In other words, for ΔS₁₀, Equation (5-2) below holds and ΔS₁₀ corresponds to the model difference.

$$Δ {S}_{10} = α \cdot \{ log {V}_{Bt} ( f ) - log {V}_{AT} ( f ) \} + α \cdot \{ log {δ}_{B} ( f ) - log {δ}_{A} ( f ) \} + α \cdot log \{ \begin{matrix}Difference between design values of \\ A and B for another factor\end{matrix} \} \qquad ( 5 \text{-} 2 )$$

As described above, the reference specimen and the ultrasound endoscope P₀ to be combined are common and thus ΔS₁₀ corresponds to the model difference of the ultrasound observation apparatus 3. The same applies to the case where the common ultrasound endoscope to be combined is replaced with P₁. A difference ΔS₁₁ between both sets of reference spectral data is defined by Equation (6-1) below.

ΔS₁₁=S(PᵢB₀; f, z)−S(PᵢA₀; f, z) (6-1)

For the difference ΔS₁₁ between both the sets of reference spectral data defined by Equation (6-1), Equation (6-2) below holds because of the same reason as that for Equation (5-2).

$$Δ {S}_{11} = α \cdot \{ log {V}_{Bt} ( f ) - log {V}_{AT} ( f ) \} + α \cdot \{ log {δ}_{B} ( f ) - log {δ}_{A} ( f ) \} + α \cdot log \{ \begin{matrix}Difference between design values of \\ A and B for another factor\end{matrix} \} \qquad ( 6 \text{-} 2 )$$

The right sides of Equation (5-2) and Equation (6-2) are equal to each other and therefore Equation (6-3) below holds.

ΔS₁₀=ΔS₁₁ (6-3)

Thus, the differences ΔS₁₁ and ΔS₁₁ each between sets of reference spectral data are equal to each other and corresponds to the model difference of the ultrasound observation apparatus 3.

Equation (3-1) is derived from the above-described Equation. First of all, Equation (6-4) is obtained by assigning Equation (5-1) and Equation (6-1) to Equation (6-3).

S(PᵢB₀; f, z)=S(P₀B₀; f, z)−S(P₀A₀; f, z)+S(PᵢA₀; f, z) (6-4)

According to Equation (4), the left side of Equation (6-4) is equal to S(PᵢBₘ; f, z) and thus Equation (3-1) is led.

It is obvious that, even when the common observation subject is changed from the reference specimen to tissue in a human body, Equations (5-2), (6-2) and (6-3) hold. In other words, the model difference affecting the reference spectral data obtained from the reference specimen and the model difference affecting the subject spectral data obtained from the common observation subject in the human body are equal in value to each other. Accordingly, it can be concluded that correcting the subject spectral data based on the model difference that is calculated by Equation (3-1) is rational.

The individual difference of ultrasound endoscopes and the individual difference between the individual P₀ and the individual Pᵢ of Model P will be looked at below. Assume that the reference individual A₀ of Model A of the ultrasound observation apparatus 3 is connected to each of the individual P₀ serving as the reference individual of Model P and the individual Pᵢ and reference spectral data S(P₀A₀; f, z) and S(PᵢA₀; f, z) are obtained from a reference specimen. As described above, as the factor affecting the subject spectral data, the sensitivity difference of the ultrasound transducer 21, its frequency characteristic difference, and the frequency characteristic difference of wiring, such as cables incorporated in the insertion unit of the ultrasound endoscope 2, are exemplified and variations thereof appear as an individual difference in the ultrasound endoscope. For example, when γ₀(f) is set for frequency characteristics of sensitivity in the individual P₀ and s₀(f) is set for the frequency characteristics of wiring. The frequency component V(f, L) of voltage amplitude serving as the basis of the reference spectral data S(P₀A₀; f, z) contains γ₀(f) and ε₀(f) in the multiplying factor. As for the ultrasound endoscope 2, even when there is another factor of effect on the reference spectral data S(P₀A₀; f, z), the design value serving as the basis of the factor is generally contained as the multiplication factor of V(f, L).

According to Equation (1), calculation of S(P₀A₀; f, z) uses a common logarithm operation of V(f, L) and thus all these factors are contained as terms of addition forming S(P₀A₀; f, z). In other words, S(P_{C}A₀; f, z) contains a term of addition of α·log γ₀(f)+α·log ε₀(f)+α·log (another factor). Eventually, as for the ultrasound endoscope 2, the design value serving as the basis of the cause of effect on the reference spectral data S(P₀A₀; f, z) is contained as the term of addition of S(P₀A₀; f, z) itself.

Similarly, for example, γᵢ(f) is set for frequency characteristics of sensitivity in the individual Pᵢ and εᵢ(f) is set for frequency characteristics of wiring. As for the ultrasound endoscope 2, the design value serving as the basis of the cause of effect on the reference spectral data S(PᵢA₀; f, z) contains a term of addition of α·log γᵢ(f)+α·log εᵢ(f)+α·log (another factor).

A difference of ΔS₂₀ of both sets of reference spectral data is defined by Equation (7-1) below.

ΔS₂₀=S(PᵢA₀; f, z)−S(P₀A₀; f, z) (7-1)

The reference specimen and the ultrasound observation apparatus A₀ to be combined is common and thus common terms are offset in the process of subtraction of Equation (7-1) and the variations described above are obtained. In other words, for ΔS₂₀, Equation (7-2) below holds and ΔS₂₀ corresponds to the individual difference.

$$Δ {S}_{20} = α \cdot \{ log {γ}_{i} ( f ) - log {γ}_{0} ( f ) \} + α \cdot \{ log {ɛ}_{i} ( f ) - log {ɛ}_{0} ( f ) \} + α \cdot log \{ \begin{matrix}Difference between design values of \\ {P}_{i} and {P}_{0} for another factor\end{matrix} \} \qquad ( 7 \text{-} 2 )$$

As described above, the reference specimen and the ultrasound observation apparatus Δ₀ to be combined are common and thus ΔS₂₀ corresponds to the individual difference of the ultrasound endoscope 2. The same also applies to the case where the common ultrasound observation apparatus to be combined is replaced with B₀. A difference ΔS₂₁ between both sets of reference spectral data is defined by Equation (8-1) below.

ΔS₂₁=S(PᵢB₀; f, z)−S(P₀B₀; f, z) (8-1)

Furthermore, for the difference ΔS₂₁ between sets of reference spectral data defined by Equation (8-1), Equation (8-2) below holds because of the same reason for Equation (7-2).

$$Δ {S}_{21} = α \cdot \{ log {γ}_{i} ( f ) - log {γ}_{0} ( f ) \} + α \cdot \{ log {ɛ}_{i} ( f ) - log {ɛ}_{0} ( f ) \} + α \cdot log \{ \begin{matrix}Difference between design values of \\ {P}_{i} and {P}_{0} for another factor\end{matrix} \} \qquad ( 8 \text{-} 2 )$$

The right sides of Equation (7-2) and Equation (8-2) are equal to each other and therefore Equation (8-3) below holds.

ΔS₂₀=ΔS₂₁ (8-3)

Thus, the differences ΔS₂₀ and ΔS₂₁ each between sets of reference spectral data are equal to each other and corresponds to the individual difference of the ultrasound endoscope 2.

Equation (3-1) can be derived from the above-described Equation. First of all, Equation (8-4) below is obtained by assigning Equation (7-1) and Equation (8-1) to Equation (8-3).

S(PᵢB₀; f, z)=S(P_{C}B₀; f, z)−S(A₀A₀; f, z)+S(PᵢA₀; f, z) (8-4)

According to Equation (4), the left side of Equation (8-4) is equal to S(PᵢBₘ; f, z) and thus Equation (3-1) is led.

It is obvious that, even when the common observation subject is changed from the reference specimen to tissue in a human body, Equations (7-2), (8-2) and (8-3) hold. In other words, the model difference affecting the reference spectral data obtained from the reference specimen and the value of the model difference affecting the subject spectral data obtained from the common observation subject in the human body are equal in value to each other. Accordingly, it can be concluded that correcting the subject spectral data based on the model difference that is calculated by Equation (3-1) is rational.

Furthermore, A₀, B₀ and P_{C} are reference individuals and thus, Equation (3-1) represents that a model difference is correctable using the reference spectral data obtained from a combination of a reference individual and a non-reference individual and reference spectral data that is obtained from a combination of reference individuals. Both the sets of reference spectral data are measurable in a factory, or the like, before shipping to a facility.

The reason why Equation (3-2) holds will be described below. From (3-1),

$$\begin{matrix}S ( {P}_{i} {B}_{m} ; f , z ) = S ( {P}_{0} {A}_{0} ; f , z ) + \\ S ( {P}_{0} {B}_{0} ; f , z ) - S ( {P}_{0} {A}_{0} ; f , z ) + \\ S ( {P}_{i} {A}_{0} ; f , z ) - S ( {P}_{0} {A}_{0} ; f , z ) \\ = S ( {P}_{0} {A}_{0} ; f , z ) + Δ {S}_{10} + Δ {S}_{20}\end{matrix} \qquad \\$$

(Definitional equation (5-1) of Δ₁₀ and definitional equation (7-1) of Δ₂₀ are assigned.)

Accordingly, Equation (3-2) is led.

FIG. 5 will be further described. Assume that, on a plane in FIG. 5, the length of each of the sides is defined as a difference between sets of reference spectral data. From Equations (5-1), (6-1), (6-3), (7-1), (8-1) and (8-3), the lengths of the four sides are equal to ΔS₁₀, ΔS₁₁, ΔS₂₀ and ΔS₂₁, respectively. Furthermore, from Equations (6-3) and (8-3), opposed two sides ΔS₁₀ and ΔS₁₁ have equal lengths and opposed two sides ΔS₂₀ and ΔS₂₁ have equal lengths and are not inconsistent with the definition of oblong. In other words, even when the difference between the sets of reference spectral data is assumed as a length and the arrows representing the differences at the four points illustrated in FIG. 5 are assumed as ΔS₁₀, ΔS₁₁, ΔS_{2C} and ΔS₂₁, the rectangle of the conceptual diagram is consistently an oblong and thus it can be considered that the assumption comes into effect.

As described above, from Equation (3-1) or Equation (3-2), using sets of spectral data S(P₀A₀; f, z) and S(P₀B₀; f, z) that can be acquired using reference individuals at a factory, or the like, and spectral data S(PᵢA₀; f, z) that can be acquired using the ultrasound observation apparatus that is a reference individual of Reference Model A (the ultrasound observation apparatus A₀ herein) and each individual Pᵢ of the ultrasound endoscope in a factory, or the like, also before shipping in advance, it is possible to calculate reference spectral data S(PᵢBₘ; f, z) from a combination of a given individual of ultrasound observation apparatus (ultrasound observation apparatus Bₘ) of a model different from the reference model (Model B herein) and a given individual Pᵢ of ultrasound endoscope.

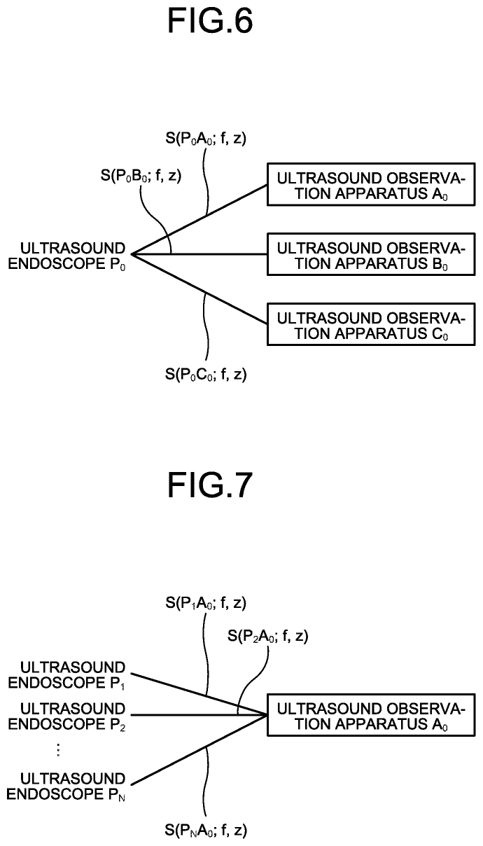

FIGS. 6 and 7 are diagrams illustrating sets of spectral data that are acquired in advance. When ultrasound endoscopes of Type P (P₁, P₂, . . . , P_{N}) are connected to ultrasound observation apparatuses of Types A, B and C, in a factory, or the like, using the ultrasound endoscope P₀ that is a reference individual of Type P and the ultrasound observation apparatuses A₀, B₀ and C₀ that are reference individuals of Types A, B and C, sets of spectral data S(P₀A₀; f, z), S(P_{C}B₀; f, z) and S(P₀C₀; f, z) based on echo signals from the reference specimen are acquired in advance (see FIG. 6). Accordingly, model difference correction spectral data for correcting the model difference is acquired.

Using each individual of Type P (ultrasound endoscopes P₁, P₂, . . . , P_{N}) and the ultrasound observation apparatus A₀ that is the reference individual of Reference Model A, sets of spectral data S(P₁A₀; f, z), S(P₂A₀; f, z), . . . , S(P_{N}A₀; f, z) are acquired in advance (see FIG. 7). Accordingly, individual difference correction spectral data for correcting the individual difference is acquired.

As a reference specimen that is used to acquire model difference correction spectral data and individual difference correction spectral data, a common phantom obtained by uniformly mixing known scatterers whose material, mass density, acoustic velocity, acoustic impedance, diameter and number density are known into a medium whose material, mass density, acoustic velocity and acoustic impedance are also known. An acryl board may be used as the reference specimen. When a phantom is used as the reference specimen, spectral data is generated based on echoes caused by backscattering. When an acryl board is used as the reference specimen, spectral data is generated based on echoes caused by total reflection (0% transparent wave and 100% backscattering).

The acquired model difference correction spectral data and the individual difference correction spectral data are stored in various storage media (the storage 37 and a hospital server 101, a factory server 102, an optical drive 103, an universal serial bus (USB) memory 104 to be described below, etc.).

The spectral corrector 314 calculates the reference spectral data S(PᵢBₘ; f, z) based on Equation (3-1) or Equation (3-2) using the model difference correction spectral data and the individual difference correction spectral data that are generated previously and further calculates the normal spectral data S_{c}(f, L) by subtracting the reference spectral data S(PᵢBₘ; f, z) from the subject spectral data S(LB; f, z).

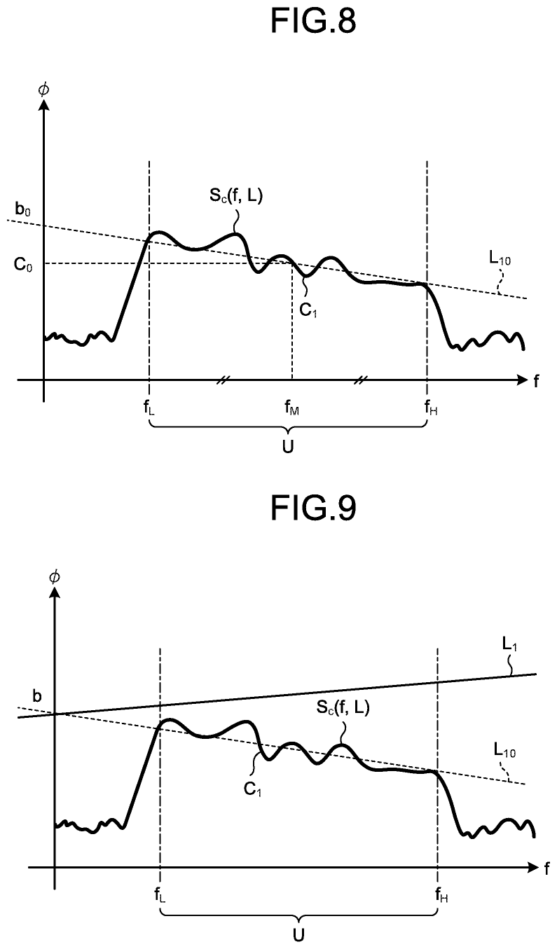

FIG. 8 is a diagram illustrating exemplary normal spectral data that is calculated by the spectral corrector 314. In FIG. 8, the horizontal axis represents the frequency f. In FIG. 8, the vertical axis represents φ and illustrates a function φ=S_{c}(f, L) using the normal spectral data S_{c}(f, L) that is given by Equation (1). The straight line (a regression line L₁₀) represented in FIG. 8 will be described below. In the first embodiment, a curved line and a straight line consist of sets of discrete points.

In spectral data C₁ represented in FIG. 8, a lower limit frequency f_{L} and an upper limit frequency f_{H} are parameters that are determined based on the frequency band of the ultrasound transducer 21, the frequency band of the pulse signal that is transmitted by the transmitter-receiver 311, etc. The frequency band that is determined by the lower limit frequency f_{L} and the upper limit frequency f_{H} in FIG. 8 will be referred to as a “frequency band U”.

The normal feature data calculator 315 calculates feature data of the normal spectral data (hereinafter, pre-correction feature data) by approximating multiple sets of normal spectral data that are output from the spectral corrector 314 by a straight line and correcting attenuation dependent on the frequency with respect to the pre-correction feature data.

The normal feature data calculator 315 performs simple linear regression analysis on the spectral data in a given frequency band to approximate the spectral data by a linear expression (regression line), thereby calculating the pre-correction feature data that characterizes the approximated linear expression. The simple linear regression analysis is regression analysis in the case where there is only one type of independent variable. The independent variable of the simple linear regression analysis in the first embodiment corresponds to the frequency f. For example, when the spectral data is in the state of the spectral data C₁ illustrated in FIG. 8, the normal feature data calculator 315 performs a simple linear regression analysis on the frequency band U and acquires the regression line L₁₀ of the spectral data C₁. The normal feature data calculator 315 then calculates, as the pre-correction feature data, a slope a₀ of the regression line L₁₀, an intercept b₀ and a mid-band fit c₀=a₀f_{M}+b₀ that is a value on the regression line of the center frequency (that is, “mid-band”) f_{M}−(f_{L}+f_{H})/2 of the frequency band U. Expressing the spectral data C₁ by the parameters of the liner formula (the slope a₀, the intercept b₀ and the mid-band fit c₀) that characterize the regression line L₁₀ as described above approximates the spectral data C₁ to the linear formula.

It is considered that, from among the three types of pre-correction feature data, the slope a₀ and the intercept b₀ have correlations with the size of the scatterers that scatter ultrasound, the intensity of scattering by the scatterers, the number density (concentration) of the scatterers, etc. The mid-band fit c₀ gives the intensity of spectrum at the center of the effective frequency band. For this reason, it is considered that the mid-band fit c₀ has a correlation with the luminance of the B-mode image to some extent in addition to the size of the scatterers, the scattering intensity of the scatterers, and the number density of the scatterers. The normal feature data calculator 315 may approximate the spectral data by regression analysis using second-degree or higher polynomial.

Correction made by the normal feature data calculator 315 will be described below. In general, the amplitude of ultrasound is attenuated exponentially with respect to the travel distance. Accordingly, when the amplitude is logarithmically transformed into a common logarithm and expressed by decibel, the amplitude attenuates linearly with respect to a round-trip distance L and attenuates linearly with respect to a reception depth z (=L/2) that achieves a round-trip distance of L. Thus, under the expression of amplitude in decibel, an attenuation amount A(f, z) occurring during the round trip of ultrasound between the reception depth 0 and the reception depth z can be expressed as a liner change between amplitudes before and after the round-trip of ultrasound (difference in decibel expression). It is known that the attenuation amount A(f, z) of amplitude depends on the frequency when the observation subject is a living body, the attenuation is large at a high frequency and the attenuation is small at a low frequency. Particularly, in uniform tissue, it is known that the attenuation is proportional to the frequency, which is expressed by Equation (9) below.

A(f, z)=2ζzf (9)

where the proportionality constant ζ is an amount referred to as an attenuation rate, z is a reception depth of ultrasound, and f is a frequency. A specific value of the attenuation rate ζ is determined according to, when the observation subject is a living body, the site in the living body and the tissue and is approximately 0.55 dB/cm/MHz for a normal liver. In the first embodiment, the value of the attenuation rate ζ is stored in the storage 37 previously and the normal feature data calculator 315 reads the value of the attenuation rate ζ from the storage 37 as appropriate and uses the value. When the ultrasound observation apparatus 3 receives an input of a site name and a tissue name of the observation subject from the technologist in advance before the ultrasound endoscope 2 transmits ultrasound, the normal feature data calculator 315 reads an appropriate value of the attenuation rate ζ corresponding to the site name and the tissue name and uses the value for the following attenuation correction. Furthermore, when the ultrasound observation apparatus 3 receives the value of the attenuation rate ζ directly from the technologist, the normal feature data calculator 315 users the value for the following attenuation correction. When the ultrasound observation apparatus 3 receives no input from the technologist, the normal feature data calculator 315 uses the above-mentioned 0.55 dB/cm/MHz for the following attenuation correction.

The normal feature data calculator 315 performs attenuation correction on the extracted pre-correction feature data (the slope a₀, the intercept b₀ and the mid-band fit c₀) according to Equations (10) to (12) represented below, thereby calculating sets of post-correction feature data a, b and c (normal feature data below).

a=a₀+2ζz (10)

b=b₀ (11)

c=c₀+A(f_{M}, z)=c₀+2ζzf_{M}(=af_{M}+b) (12)

As it is clear from Equations (10) and (12), the greater the reception depth z of ultrasound is, the greater the normal correction amount by which the normal feature data calculator 315 performs correction is. According to Equation (11), correction on the intercept is identity transformation. This is because the intercept is a frequency component corresponding to a frequency of 0 and thus is not affected by attenuation.

FIG. 9 is a graph representing a straight line having, as parameters, the sets of normal feature data a, b and c that are calculated by the normal feature data calculator 315. Taking the vertical axis in FIG. 9 as φ, a straight line L₁ is expressed by Equation (13) below.

ϕ=af+b=(a₀+2ζz)f+b₀ (13)

As it is clear from Equation (13), the straight line L₁ has a slope greater than that of the regression line L₁₀ before attenuation correction (a>a₀) and has an intercept equal to that of the regression line L₁₀. The normal feature data calculator 315 then outputs the resultant sets of normal feature data a, b and c obtained by performing attenuation correction to the feature data image data generator 316.

Back to FIG. 1, the feature data image data generator 316 generates feature data image data obtained by assigning visual information relative to the normal feature data, which is calculated by the normal feature data calculator 315, correspondingly to each pixel of the image of the B-mode image data. To the pixel area corresponding to the data amount of the single RF data string Fⱼ (j=1, 2, . . . , K), the feature data image data generator 316 assigns the visual information relative to the normal feature data of frequency spectrum that is calculated from the RF data string Fⱼ. The visual information relative to the feature data, for example, variables of a color space forming a given color system of hue, chroma, brightness, luminance, R (red), G (green), B (blue), etc., can be exemplified.

The synthesizer 317 synthesizes the B-mode image data that is generated by the B-mode image data generator 312 and the feature data image data that is generated by the feature data image data generator 316 to generate synthesized image data that is obtained by superimposing the visual information relative to the feature data onto each pixel of the image of the B-mode image data.

The frequency analyzer 313, the spectral corrector 314, the normal feature data calculator 315, the feature data image data generator 316 and the synthesizer 317 may perform the above-described respective sets of processing with a limitation of the area to be analyzed to a region of interest (ROI) that is sectioned by a specific depth width and a specific orientation width (that is, width in the scanning direction) from among the scanned area S illustrated in FIG. 3. Limiting the area of interest to a necessary area makes it possible to reduce the operational amount and improve the rate ζ for display. The case where the area of interest is limited in the first embodiment will be described below.

The functions of the respective units of the ultrasound observation apparatus 3 excluding the image generator 31 and various input-output devices and a server will be described below.

A keyboard 105 is formed of a plurality of buttons with which various types of information can be input and he keyboard 105 receives inputs from the technologist. The keyboard 105 is provided with a touch panel 105a having a display screen. The touch panel 105a, for example, receives an input corresponding to the positions of contact of fingers of the technologist. Then, according to operation icons displayed on the display screen on the touch panel 105a, the keyboard 105 then outputs an operation signal containing the positions (sets of coordinates) of touch (contact) made by the technologist and the button numbers that identity the buttons to which inputs are made to the keyboard input receiver 36. The touch panel 105a displays the ultrasound image and various types of information and thus functions as a graphical user interface (GUI). There are resistive, capacitive, and optical touch panels and any type of touch panel may be used.

The keyboard input receiver 36 generates a choice signal containing information representing which key and which menu is chosen and input according to the operation signal from the keyboard 105 and outputs the choice signal to the external communication controller 33.

According to the content of the choice signal from the keyboard input receiver 36, as required, the external communication controller 33 generates a combination model number data in which the models and individuals of the ultrasound endoscope 2 and the ultrasound observation apparatus 3 are associated and outputs the combination model number data to the write-read unit 32. Specifically, the combination model number data is data in which model names and individual numbers (generally referred to as serial numbers) are associated. The external communication controller 33 outputs the choice signal to the write-read unit 32 when required, which will be described below.

Based on a read instruction from the write-read unit 32, the external communication controller 33 chooses a communication unit for connection to acquire the reference spectral data from the network communication unit 34 and the device communication unit 35 and outputs the combination model number data and the read instruction to the chosen communication unit to cause the chosen communication unit to read the reference spectral data.

The write-read unit 32 performs a read process of, if required, according to the content of the choice signal from the external communication controller 33, reading the reference spectral data (containing the above-described model difference correction spectral data and the individual difference correction spectral data) suitable to the content of the choice signal from the storage 37. When the corresponding reference spectral data is not stored in the storage 37, the write-read unit 32 outputs a read instruction to the external communication controller 33 to cause the external communication controller 33 to read the reference spectral data. The functions of the external communication controller 33 after the output of the read instruction are as described above.

The network communication unit 34 transmits the combination model number data, for example, to the hospital server 101 in a hospital via the communication network mentioned above and acquires the reference spectral data corresponding to the combination model number data. The network communication unit 34 may acquires the reference spectral data from the factory server 102 via the Internet from the hospital server 101.

The device communication unit 35 acquires the reference spectral data corresponding to the combination model number data by communicating with the device that is connected to the ultrasound observation apparatus 3, such as the optical drive 103 or the USB memory 104. The optical drive 103 is achieved using, for example, a CD drive or a DVD drive.

The storage 37 includes a memory 371a and a hard disk drive (HDD) 371b that store sets of feature data that are calculated for each frequency spectrum by the normal feature data calculator 315, the image data that is generated by the B-mode image data generator 312, the feature data image data generator 316 and the synthesizer 317, operational parameters and data for each process.

The HDD 371b further sores, in addition to the above-listed information, for example, information, such as information necessary for amplification (the relationship between the amplification factor and the reception depth), information necessary for logarithmic transformation (refer to Equation (1), for example, the values of α and V_{c}), and a window function necessary for frequency analysis (such as Hamming, Hanning or Blackman).

The storage 37 includes, as an additional memory, a read only memory (ROM) not illustrated in the drawings in which an operation program for executing the operation method of the ultrasound observation apparatus 3 is installed in advance. The operation program may be also recordable in a computer-readable recording medium, such as a portable hard disk, a flash memory, a CD-ROM, a DVD-ROM or a flexible disk, and thus may be widely distributable. The above-described various programs can be acquired by being downloaded via a communication network. The communication network herein is achieved with, for example, an existing public network, a LAN, a WAN, or the like, and it does not matter whether the communication network is wired or wireless.

The controller 38 is achieved using a general-purpose processor, such as a CPU having arithmetic and control functions, a dedicated integrated circuit, such as an ASIC or a FPGA, or the like. The controller 38 reads information, such as the operation program, the arithmetic parameters and the data for each process, that the storage 37 stores, etc., from the storage 37 via the write-read unit 32 and executes various arithmetic processes relating to the operation method of the ultrasound observation apparatus 3, thereby overall controls the ultrasound observation apparatus 3. The controller 38 may be formed using a general-purpose processor that is shared by the image generator 31 or a dedicated integrated circuit.

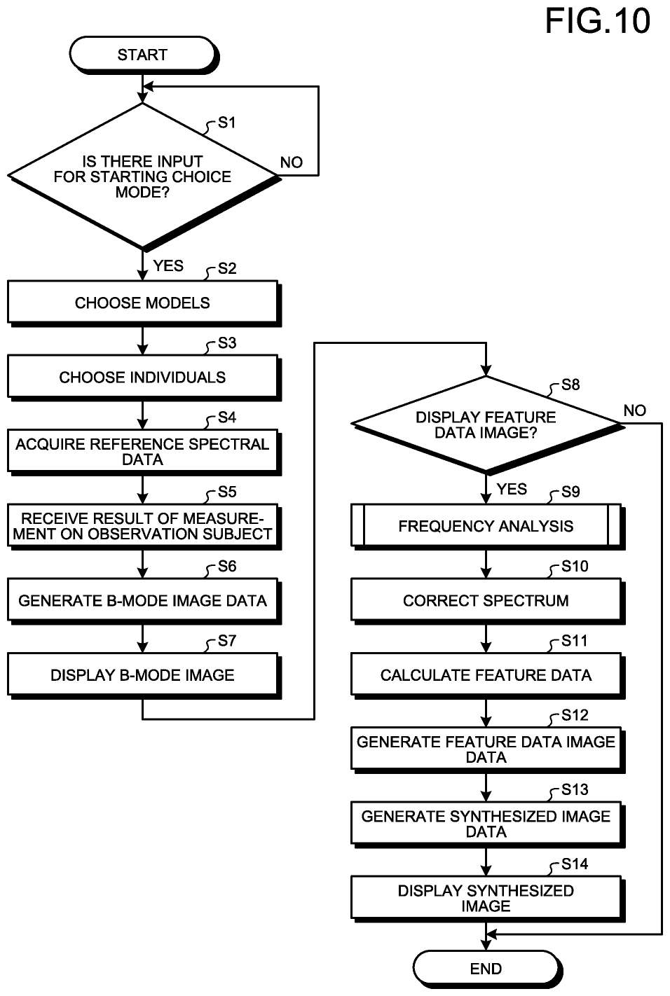

FIG. 10 is a flowchart illustrating an overview of the process performed by the ultrasound observation apparatus 3 having the above-described configuration. Herein, the case where a facility that a technologist belongs, such as a hospital, already possesses the ultrasound endoscopes 2 (the individuals P₁ and P₂) of Type P and the ultrasound observation apparatus 3 of Type A and the observation apparatus 3 of Type B is newly purchased will be assumed and described. The overview is a function necessary to, according to operations of the technologist, specify necessary reference spectral data, download the reference spectral data, and correct subject spectral data to normal spectral data using the reference spectral data.

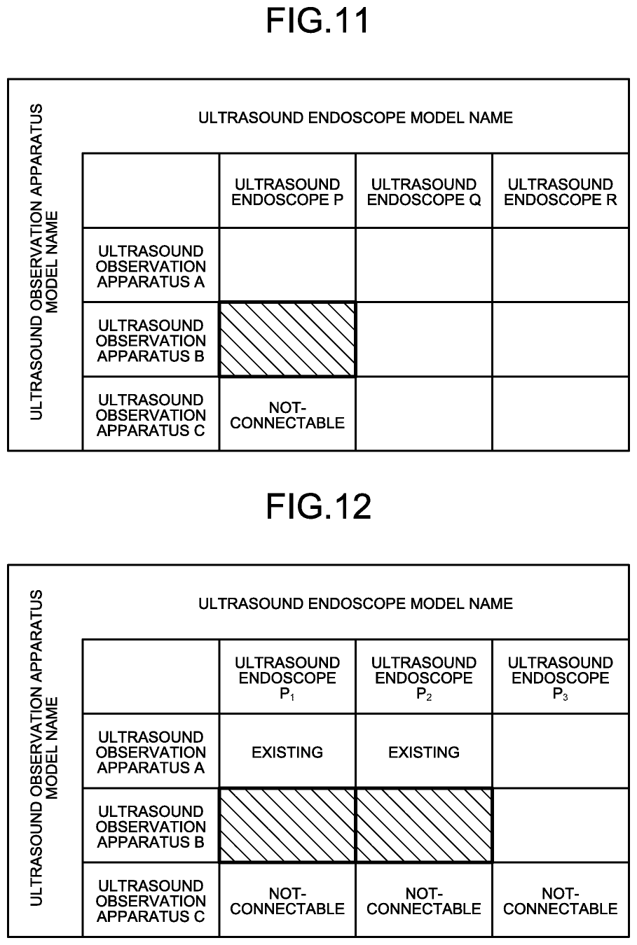

At step S1, first of all, the external communication controller 33 determines whether there is an input of a choice signal for entering a choice mode for acquiring reference spectral data from the keyboard input receiver 36. The choice mode is a mode of the user interface for specifying a model and an individual of the ultrasound observation apparatus to be described below and, in the choice mode, a model choice screen illustrated in FIG. 11 and an individual choice screen illustrated in FIG. 12 are displayed. When there is an input of a choice signal for starting the choice mode to the external communication controller 33 (YES at step S1), the ultrasound observation apparatus 3 moves to step S2. On the other hand, when there is no input of the choice signal for starting the choice mode to the external communication controller 33 (NO at step S1), the ultrasound observation apparatus 3 repeats choice signal checking.

At step S2, the external communication controller 33 outputs a read instruction to read a model list and connectability information to the write-read unit 32. The write-read unit 32 searches the storage 37 and reads the model list of the ultrasound observation apparatus 3, the model list of the ultrasound endoscope 2, and the connectability information on various models that are stored in the storage 37 and outputs the model lists and the connectability information to the external communication controller 33. The external communication controller 33 generates a model choice screen to choose models of the ultrasound observation apparatus 3 and the ultrasound endoscope 2 based on the model lists and the connectability information and causes the touch panel 105a of the keyboard 105 to display the model choice screen via the keyboard input receiver 36. In this manner, the choice mode starts. The model lists are downloadable from the network communication unit 34, the hospital server 101, and the factory server 102 and updatable to lists of latest models on sale.

FIG. 11 is a diagram illustrating the model choice screen to choose models of the ultrasound endoscope 2 and the ultrasound observation apparatus 3. As illustrated in FIG. 11, models of the ultrasound observation apparatus 3 and models of the ultrasound endoscope 2 are displayed on the model choice screen. In FIG. 11, for explanation, models are expressed by A, B, C, P, Q and R on the model choice screen; however, model names are displayed practically. On the model choice screen, a combination of models that are not connectable according to the connectability information is displayed by texts of “not-connectable”. The technologist touches a rectangle (for example, the hatched area in FIG. 11) corresponding to the corresponding combination according to the models that are set in the facility and the combination to be used. Touching multiple rectangles enables multiple choices. The keyboard 105 outputs, as an operation signal, coordinate information corresponding to the position of contact on the touch panel 105a to the keyboard input receiver 36. The keyboard input receiver 36 specifies a combination of the ultrasound observation apparatus model and the ultrasound endoscope model corresponding to the chosen rectangle and outputs the information as a choice signal to the external communication controller 33. Accordingly, information on the model of the ultrasound endoscope 2 and the model of the ultrasound observation apparatus 3 is input to the external communication controller 33. When the technologist touches a menu representing that choosing models has ended, the ultrasound observation apparatus 3 moves to step S3. For example, when the technologist touches the single hatched area in FIG. 11 and the process ends, Model P of the ultrasound endoscope and Model B of the ultrasound observation apparatus are chosen.

At step S3, the write-read unit 32 searches the storage 37, generates a list of sets of reference spectral data stored in the storage 37 (simply referred to as “reference spectral data list” below) and outputs the reference spectral data list to the external communication controller 33. In the reference spectral data list, the model names and the individual numbers of the ultrasound endoscope 2 and the ultrasound observation apparatus 3 serving as the basis of the sets of reference spectral data are associated with the file names of the respective sets of reference spectral data. The external communication controller 33 generates an individual choice screen to choose individuals of the ultrasound endoscope 2 based on the reference spectral data list and causes the touch panel 105a of the keyboard 105 to display the individual choice screen via the keyboard input receiver 36.

FIG. 12 is a diagram illustrating the individual choice screen to choose individuals of the ultrasound endoscope 2. As illustrated in FIG. 12, the models of the ultrasound observation apparatus 3 and the individuals of the ultrasound endoscope 2 are displayed on the individual choice screen. In FIG. 12, for explanation, the models are expressed by A, B, and C and the individuals are expressed by P₁, P₂ and P₃ on the individual choice screen; however, model names are displayed as models and individual numbers are displayed as individuals practically. FIG. 12 represents the individual choice screen in the example where the technologist touches the single hatched area in FIG. 11 and the process ends, and the individuals P₁, P₂ and P₃ of Model P of the ultrasound endoscope and Model B of the ultrasound observation apparatus are displayed. On the individual choice screen, reference spectral data combinations that are already stored from the reference spectral data list are displayed by characters of “existing”. According to the combinations of the individual numbers of the ultrasound endoscopes possessed by the facility and the model of the ultrasound observation apparatus to which the ultrasound endoscopes are connected, the technologist touches rectangles corresponding to the corresponding combinations (for example, the hatched areas in FIG. 12). Touching multiple rectangles enables multiple choices. The keyboard 105 outputs coordinate information corresponding to the positions of contact on the touch panel 105a as an operation signal to the keyboard input receiver 36. The keyboard input receiver 36 specifies combinations each of a model of an ultrasound observation apparatus and a model and an individual of an ultrasound endoscope, which are combinations corresponding to the chosen rectangles, and outputs information of the combinations as a choice signal to the external communication controller 33. Accordingly, information on the individuals of the same model of the ultrasound endoscope 2 and the model of the ultrasound observation apparatus 3 is input to the external communication controller 33. When the technologist touches a menu representing that choosing individuals has ended, the ultrasound observation apparatus 3 moves to step S4. For example, when the technologist touches the two hatched areas in FIG. 12 and the process ends, the individuals P₁ and P₂ of Model P of ultrasound endoscope and Model B of ultrasound observation apparatus are chosen. The choice mode ends here.

When information on models and individuals is input on the individual choice screen, the external communication controller 33 generates combination model data containing the information on the model of the ultrasound endoscope 2 and the model of the ultrasound observation apparatus 3 and the information on the individuals of the ultrasound endoscope 2 and the model of the ultrasound observation apparatus 3 and outputs the combination model number data to the write-read unit 32.

At step S4, the write-read unit 32 acquires the combination model number data, acquires the reference spectral data from the storage 37 or reads the reference spectral data by performing control to cause at least any one of the network communication unit 34 and the device communication unit 35 to acquire the reference spectral data relating to the chosen models and individuals, and inputs the reference spectral data to the spectral corrector 314. When the corresponding reference spectral data is not stored in the storage 37, the write-read unit 32 causes at least any one of the network communication unit 34 and the device communication unit 35 to read the reference spectral data via the external communication controller 33. The spectral data acquired herein is the reference spectral data S(PᵢB₀; f, z) that is calculated in advance or, for example, the spectral data S(P₀A₀; f, z), the spectral data S(P₀B₀; f, z) and the spectral data S(PᵢA₀; f, z) using the reference individual A₀ of Reference model A of the ultrasound observation apparatus. Description will be given below, assuming that the spectral data S(P₀A₀; f, z), the spectral data S(P₀B₀; f, z), and the spectral data S(PᵢA₀; f, z) using the reference individual A₀ of Reference model A of the ultrasound observation apparatus for calculating the reference spectral data are acquired.

Steps S1 to S4 are executed when the ultrasound observation apparatus 3 is started for the first time or when the choice mode to specify models and individuals is started via the keyboard 105, or the like. When the ultrasound observation apparatus 3 is started for the second time or later or when the choice mode is not started, the ultrasound observation apparatus 3 executes the process of the following steps S5 to S14.

At step S5, observation of a subject, such as tissue in a human body, starts in the facility. The ultrasound transducer 21 scans the subject and transforms the echoes that are received from the subject into an electric echo signal. The transmitter-receiver 311 receives the echo signal via the ultrasound endoscope 2. The transmitter-receiver 311 amplifies the echo signal. The transmitter-receiver 311 then samples and discretizes the echo signal that is amplified at an appropriate sampling frequency (for example, 50 MHz) to generate RF data and outputs the RF data to the B-mode image data generator 312 and the frequency analyzer 313.

At step S6, the B-mode image data generator 312 amplifies the RF data based on, for example, the relationship between the amplification factor and the reception depth (STC correction). The B-mode image data generator 312 generates B-mode image data using the RF data that is output from the transmitter-receiver 311 and outputs the B-mode image data to the synthesizer 317.

At step S7, the synthesizer 317 does not perform any processing on the B-mode image data and outputs the B-mode image data to the display device 4. The display device 4 having received the B-mode image data displays a B-mode image corresponding to the B-mode image data.

At step S8, the controller 38 checks which of “displaying” and “not displaying” the feature data image is chosen previously by the technologist via a button or a menu (not illustrated in the drawings) of the keyboard 105. When choice of “displaying” is confirmed, the controller 38 outputs a feature data image creation start command to each unit forming the image generator 31 (YES at step S8). On the other hand, when choice of “not displaying” is confirmed, the controller 38 does not output the feature data image creation start command (NO at step S8).

On receiving the feature data image creation start command, the image generator 31 performs the process at and after step S9. Regardless whether there is the feature data image creation start command, the transmitter-receiver 311 and the B-mode image data generator 312 of the ultrasounds observation apparatus 3 repeats the process from step S5 to S7 above. For this reason, while the technologist is issuing an instruction “not to display” the feature data image via the keyboard 105, the B-mode image is displayed on the display device 4 repeatedly each time the subject is scanned by the ultrasound transducer 21.

At step S9, when each unit of the image generator 31 receives the feature data image creation start command, first of all, the frequency analyzer 313 sections RF data (line data) of each sound ray at relatively short given time intervals and performs frequency analysis on the RF data of each of the sectioned blocks by FFT operation. Accordingly, spectral data (subject spectral data) corresponding to all the RF data strings is calculated (frequency analysis step).

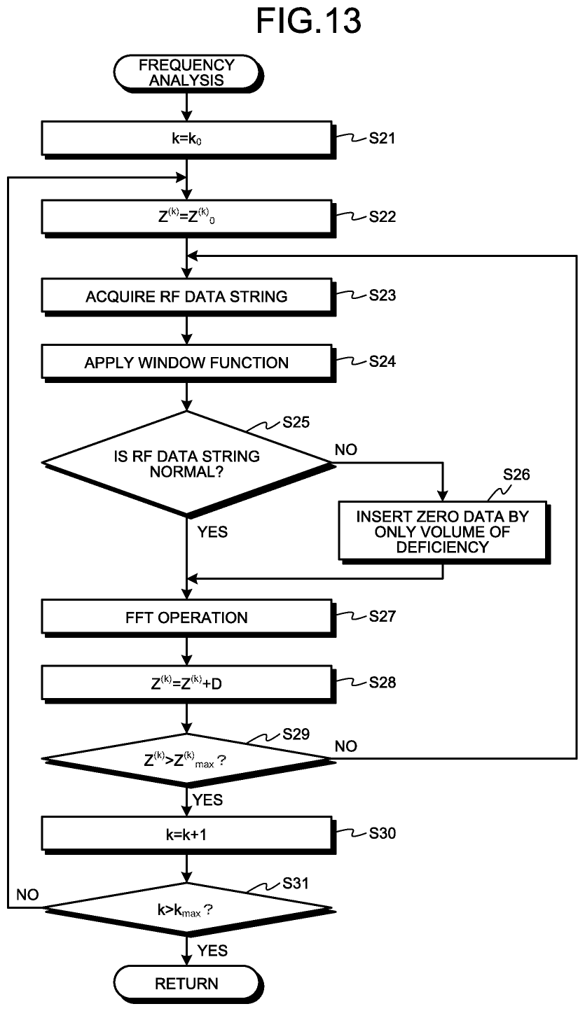

FIG. 13 is a flowchart illustrating an overview of a process that is executed by the frequency analyzer 313 at step S9. With reference to the flowchart illustrated in FIG. 13, the frequency analysis will be described in detail below.

At step S21, the frequency analyzer 313 sets k_{C} for a counter k that identifies a sound ray to be analyzed. The initial value k₀ is the number of the rightmost sound ray in the area to be analyzed in FIG. 3.

At step S22, the frequency analyzer 313 sets an initial value Z^{(k)}₀ at a data position (corresponding to the reception depth) of Z^{(k)} representing the set of RF data strings that are acquired for FFT operation. For example, FIG. 4 illustrates the case where the eighth data positon of a sound ray SRₖ is set as the initial value Z^{(k)}₀ as described above. The initial value Z^{(k)}₀ is a data position representing the shallowest RF data string in the area to be analyzed on the sound ray SRₖ.

The frequency analyzer 313 then acquires a RF data string (step S23) and applies the window function that is stored in the storage 37 to the acquired data string (step S24). Applying the window function to the RF data string as described above enables the RF data string to avoid being discontinuous at boundaries and enables prevention of occurrence of artifacts.

The frequency analyzer 313 determines whether the RF data string at a data position Z^{(k)} is a normal RF data string (step S25). As described when FIG. 4 is referred to, the RF data string has to include the number of sets of data corresponding to the power of 2. For the number of sets of data of a normal RF data string, 2ⁿ (n is a positive integer) is set below. In the first embodiment, the data position Z^{(k)} is set such that Z^{(k)} is as much as possible at the center of the RF data string to which the Z^{(k)} belongs. Specifically, because the number of sets of data of the RF data string is 2ⁿ and thus Z^{(k)} is set at the position corresponding to 2ⁿ/2 (=2ⁿ⁻¹) near the center of the RF data string. In this case, the normal RF data string means that there are 2ⁿ⁻¹−1 (−N) sets of data on a side shallower than the data position Z^{(k)} and there are 2ⁿ⁻¹ (=M) sets of data on a side deeper than the data position Z^{(k)}. In the case illustrated in FIG. 4, the RF data strings F₁, F₂, F₃, . . . , F_{K-1} are all normal. Note that FIG. 4 exemplifies the case where n=4 (N=7 and M=8).

When it is determined at step S25 that the RF data string at the data position Z^{(k)} is normal (YES at step S25), the frequency analyzer 313 moves to step S27 to be described below.

When it is determined at step S25 that, when the RF data string at the data position Z^{(k)} is not normal (NO at step S25), the frequency analyzer 313 generates a normal data string by inserting zero data by the volume of deficiency (step S26). To the RF data string that is determined not to be normal at step S25 (for example, the RF data string F_{K} in FIG. 5), the window function is applied before zero data is added. For this reason, even when zero data is inserted into the RF data string, discontinuity of data does not occur. After step S26, the frequency analyzer 313 moves to step S27 to be described below.

At step S27, by performing an FFT operation on the RF data string, the frequency analyzer 313 calculates V(f, L) corresponding to a frequency distribution of voltage amplitude of the echo signal. Thereafter, the frequency analyzer 313 performs logarithmic transformation on V(f, L), thereby obtaining spectral data S(f, L) (step S27).

At step S28, the frequency analyzer 313 changes the data position Z^{(k)} by a step width D. For the step width D, an input value that is input by the technologist via the keyboard 105 is stored in the storage 37 previously. FIG. 4 exemplifies the case where D=15.

The frequency analyzer 313 then determines whether the data positon Z^{(k)} is above a maximum value Z^{(k)}ₘₐₓ in the sound ray SRₖ (step S29). The maximum value Z^{(k)}ₘₐₓ is a data position representing the deepest RF data string in the area to be analyzed on the sound ray SRₖ. When the data positon Z^{(k)} is above the maximum value Z^{(k)}ₘₐₓ (YES at step S29), the frequency analyzer 313 increments the counter k by 1 (step S30), which means that the process shifts to the neighboring sound ray. On the other hand, when the data positon Z^{(k)} is at or under the maximum value Z^{(k)}ₘₐₓ (NO at step S29), the frequency analyzer 313 returns to step S23.

After step S30, the frequency analyzer 313 determines whether the counter k is above a maximum value kₘₐₓ (step S31). When the counter k is above the maximum value kₘₐₓ (YES at step S31), the frequency analyzer 313 ends the frequency analysis process sequence. On the other hand, when the counter k is at or under kₘₐₓ (NO at step S31), the frequency analyzer 313 returns to step S22. The maximum value kₘₐₓ is a number of the leftmost sound ray in the area to be analyzed in FIG. 3.

As described above, the frequency analyzer 313 performs the FFT operation for multiple times respectively for depths of the (kₘₐₓ−k₀+1) sound rays in the area to be analyzed. The FFT operation results are stored in the storage 37 together with reception depths and the receiving directions.

For the four types of values k₀, kₘₐₓ, Z^{(k)}₀ and Z^{(k)}ₘₐₓ, default values covering all the scanned area in FIG. 3 are stored in the storage 37 previously and the frequency analyzer 313 reads the values as appropriate and performs the process in FIG. 13. After reading the default values, the frequency analyzer 313 performs the frequency analysis process on the whole scanned area. The four types of values k₀, kₘₐₓ, Z^{(k)}₀ and Z^{(k)}ₘₐₓ are changeable according to input of an instruction about an area of interest that is made by the technologist via the keyboard 105. When the values have been changed, the frequency analyzer 313 performs the frequency analysis process only on the area of interest about which an instruction is input.

Back to FIG. 10, at step S10, following the frequency analysis process at step S9 described above, the spectral corrector 314 corrects multiple sets of spectral data that are calculated by the frequency analyzer 313. The spectral corrector 314 generates normal spectral data by Equations (2), (3-1) and (3-2) using the reference spectral data that is acquired at step S4 and the subject spectral data that is calculated at step S9. For example, the spectral corrector 314 calculates reference spectral data S(PᵢB₀; f, z) by Equation (3-1) or Equation (3-2) from the sets of spectral data S(P₀A₀; f, z) and S(P₀B₀; f, z) and the spectral data S(PᵢA₀; f, z) using a reference individual A₀ of Reference model A of ultrasound observation apparatus. The spectral corrector 314 then calculates normal spectral data Sc(LB; f, L) using Equation (2) by subtracting the reference spectral data S(PᵢBₘ; f, z) from the subject spectral data S(LB; f, z). Note that the reference spectral data S(PᵢB₀; f, z) may be calculated previously when the spectral data is acquired at step S4.

At step S11, the normal feature data calculator 315 calculates normal feature data using the normal spectral data that is generated by the spectral corrector 314. The normal feature data calculator 315 performs simple linear regression analysis on each of a plurality of sets of spectral data corresponding to positions in the area to be analyzed that is generated by the spectral corrector 314, thereby calculating pre-correction feature data corresponding respectively to the sets of spectral data. Specifically, the normal feature data calculator 315 performs single linear regression analysis on each of the sets of spectral data for approximation by a linear expression, thereby calculating a slope a₀, an intercept b₀ and a mid-band fit c₀ as pre-correction feature data. For example, the regression line L₁₀ illustrated in FIG. 8 is a regression line obtained by approximation performed by the normal feature data calculator 315 by single linear regression analysis on the spectral data C₁ of the frequency band U.

The normal feature data calculator 315 calculates post-attenuation-correction feature data by performing attenuation correction on the pre-correction feature data that is obtained by performing approximation on each set of spectral data using the attenuation rate ζ and stores the post-attenuation-correction feature data in the storage 37. The post-attenuation-correction feature data serves as normal feature data. The straight line L₁ illustrated in FIG. 9 is an exemplary straight line that is obtained by the normal feature data calculator 315 by performing the attenuation correction process.

The normal feature data calculator 315 calculates post-correction feature data a and c by assigning the data position Z=(vₛ/(2·fₛₚ)·D·n+Z₀ that is obtained using a data array of a sound ray of the ultrasound signal to the reception depth z in Equations (10 and (12), where fₛₚ is a sampling frequency of data, vₛ is an acoustic velocity, D is a data step width, n is the number of data steps from the first set of data of a sound ray to the data position of the RF data string to be processed, Z₀ is the shallowest reception depth in the area to be analyzed. For example, the sampling frequency fₛₚ of data is set at 50 MHz, the acoustic velocity is set at 1530 m/sec, and the data array illustrated in FIG. 4 is employed and the data step width D is set at 15, z=0.2295n+Z₀ (mm).

At step S12, the feature data image data generator 316 generates feature data image data obtained by assigning visual information relative to the normal feature data that is calculated by the normal feature data calculator 315 accordingly to each pixel of the image of the B-mode image data.

At step S13, the synthesizer 317 synthesizes the B-mode image data that is generated by the B-mode image data generator 312 and the feature data image data that is generated by the feature data image data generator 316, thereby generating synthesized image data obtained by superimposing visual information relative to the feature data on each pixel of the image of the B-mode image data.

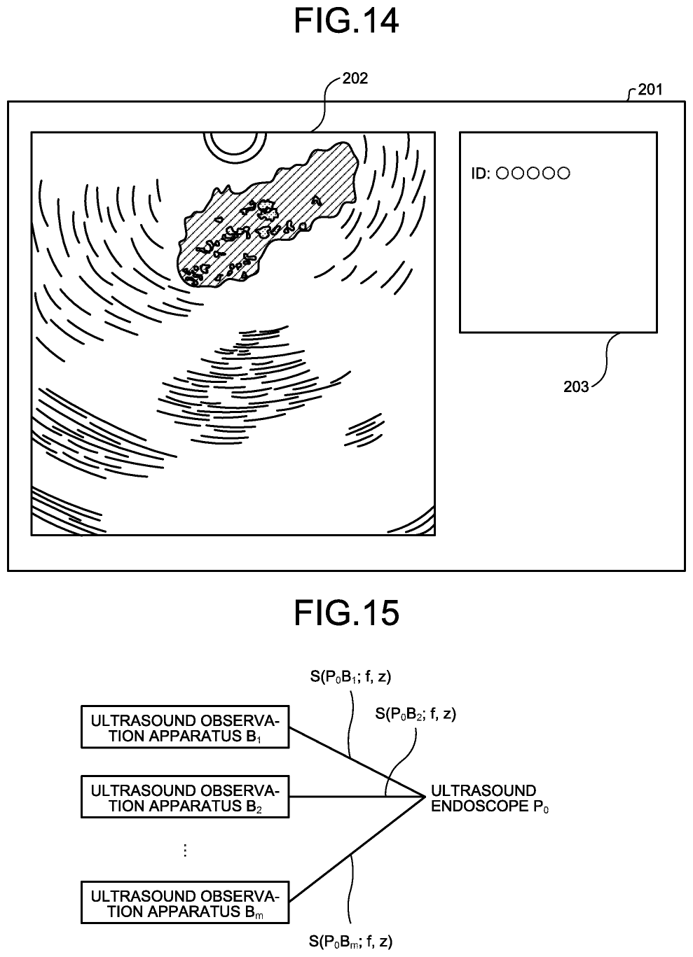

At step S14, under the control of the controller 38, the display device 4 displays the synthesized image corresponding to the synthesized image data that is generated by the synthesizer 317. FIG. 14 illustrates the exemplary display. A screen 201 illustrated in FIG. 14 includes a synthesized image display unit 202 that displays the synthesized image and an information display unit 203 that displays identification information on the observation subject. The information display unit 203 may further display information of the feature data, information of an approximation equation, information of gain, contrast, etc. Furthermore, the B-mode image corresponding to the synthesized image may be displayed in parallel with the synthesized image.

In the process sequence (steps S1 to S14) described above, the process of steps S5 to S7 and the process of steps S9 to S13 may be performed in parallel.

In the first embodiment of the disclosure described above, the normal spectral data S_{c}(LB; f, z) is calculated using the reference spectral data S(PᵢBₘ; f, z) (−S(PᵢB₀; f, z)) obtained by imaging the reference specimen for the subject spectral data S(LB; f, z) that is calculated by the frequency analyzer 313 and, from the normal spectral data, the normal feature data is calculated. According to the first embodiment of the disclosure, it is possible to obtain accurate ultrasound data regardless of a model difference and an individual difference between ultrasound probes and a model difference between ultrasound observation apparatuses.

The process of preparing reference spectral data on each model and each individual of the ultrasound endoscope 2 and each model of the ultrasound endoscope 2 and the ultrasound observation apparatus 3 is time-consuming and the volume of data that should be stored is enormous. For example, when here are 1000 individuals of one model of ultrasound endoscope and each of the individuals is connectable to any one of three models of ultrasound observation apparatus, it is necessary to acquire 3000 sets of spectral data for all combinations. Furthermore, each time a new model or individual is introduced, spectral data has to be acquired. On the other hand, according to the first embodiment, it suffices if 1003 sets of spectral data consisting of three sets of spectral data for model difference correction that are acquired from the three models of ultrasound observation apparatus and 1000 sets of spectral data for individual difference correction from combinations of individuals and a given model of ultrasound observation apparatus are acquired, and it is unnecessary to acquire spectral data on new models.

In the first embodiment, when B-mode image data is generated, the above-described correction on an individual difference between ultrasound endoscopes in sensitivity may be performed. In this case, the B-mode image data generator 312 performs correction using the above-described ΔS₂₀.

In the first embodiment, the band to be analyzed when feature data is calculated may be determined by the combination of the model (individual) of ultrasound endoscope and the model of ultrasound observation apparatus. The upper-limit frequency and lower-limit frequency of the band to be analyzed and analyzed-band information including the center frequency, band width, etc., may be stored in association with the reference spectral data in accordance with the reference spectral data and may be used for correction.

### Modification of First Embodiment

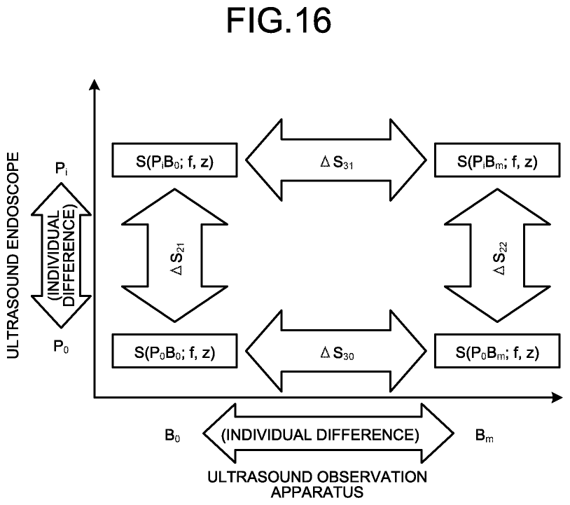

A modification of the first embodiment of the present disclosure will be described. FIGS. 15 and 16 are diagrams illustrating acquisition of reference spectral data performed by an ultrasound observation apparatus. Given that there is no individual difference of the ultrasound observation apparatus, the first embodiment has been described. In other words, given that Equation (4) holds, description has been given. Furthermore, individuals of ultrasound observation apparatuses may be corrected. From Equations (6-1), (7-1) and (6-3), Equation (14) below holds in the above-described first embodiment.

S(PᵢB₀; f, z)=S(P_{C}A₀; f, z)+ΔS_{1C}+ΔS₂₀ (14)

Equations (6-1), (7-1) and (6-3) hold even when Equation (4) does not hold and thus Equation (14) holds even when Equation (4) does not hold. Similarly, FIG. 16 holds as FIG. 5 holds. Note that ΔS₂₁ is common between FIG. 5 and FIG. 16. ΔS₂₂ is an individual difference of the ultrasound endoscope caused when replacement with an individual Bₘ of Model B of the ultrasound observation apparatus 3 is performed. Furthermore, ΔS₃₀ is an individual difference between the ultrasound observation apparatuses 3 of Model B caused when the ultrasound endoscope is an individual P₀ of Model P and ΔS₃₁ is an individual difference between the ultrasound observation apparatuses 3 of Model B caused when the ultrasound endoscope is an individual Pᵢ of Model P.

Comparing FIG. 5 and FIG. 16 with each other and considering Equation (14) similarly, Equation (15) below holds. Furthermore, Equation (16) below is an equation that defines Δ₃₀. In Modification 1, reference spectral data is acquired based on Equations (15) and (16) below.

S(PᵢBₘ; f, z)=S(P₀B₀; f, z)+ΔS₂₀+ΔS₃₀ (15)

ΔS₃₀=S(P₀Bₘ; f, z)−S(P_{C}B₀; f, z) (16)

Furthermore, assigning Equation (5-1) to Equation (15), Equation (17) below is obtained.

S(PᵢBₘ; f, z)=S(P₀A_{C}; f, z)+ΔS₁₀+ΔS₂₀+ΔS_{3C} (17)

ΔS₁₀ represents a model difference of the ultrasound observation apparatus, ΔS₂₀ represents an individual difference of the ultrasound endoscope of Model P, and ΔS₃₀ represents an individual difference of the ultrasound observation apparatus 3 of Model B.

Furthermore, Equations (5-1), (7-1) and (16) are assigned to Equation (17) and thus Equation (18) below is obtained.

$$S ( {P}_{i} {B}_{m} ; f , z ) = S ( {P}_{0} {A}_{0} ; f , z ) + S ( {P}_{0} {B}_{0} ; f , z ) - S ( {P}_{0} {A}_{0} ; f , z ) + S ( {P}_{i} {A}_{0} ; f , z ) - S ( {P}_{0} {A}_{0} ; f , z ) + S ( {P}_{0} {B}_{m} ; f , z ) - S ( {P}_{0} {B}_{0} ; f , z ) \\ ∴ S ( {P}_{i} {B}_{m} ; f , z ) = - S ( {P}_{0} {A}_{0} ; f , z ) + S ( {P}_{0} {B}_{m} ; f , z ) + S ( {P}_{i} {A}_{0} ; f , z )$$

It can be considered that the first term of Equation (18) is a model difference correction term, the second term is an individual difference correction term for the ultrasound observation apparatus 3, and the third term is an individual difference correction term for the ultrasound endoscope 2.

As described above, Equation (18) or Equation (17) represents that the model difference is correctable using reference spectral data obtained by combining the reference individuals A₀, B₀ and P_{C} and the non-reference individuals Bₘ and Pᵢ and reference spectral data obtained by combining reference individuals. Both the sets of reference spectral data are measurable in a factory, or the like, before shipping to the facility. Using both the sets of reference spectral data, it is possible to calculate reference spectral data S(PᵢBₘ; f, z) obtained by combining a given individual of ultrasound observation apparatus (the ultrasound observation apparatus Bₘ) of a model (Model B herein) different from the reference model and the given individual Pᵢ of ultrasound endoscope even when there is an individual difference of ultrasound observation apparatus.

In Modification, as for the reference spectral data S(PᵢBₘ; f, z), in addition to Δ₁₀ representing the model difference of ultrasound observation apparatus and Δ₂₀ representing the individual difference of ultrasound endoscope, Δ₃₀ representing the individual difference of the ultrasound observation apparatus 3 is taken into consideration. In Modification, as in the above-described first embodiment, it is possible to correct the ultrasound signal according to the model difference and the individual difference of the ultrasound probe and the model difference and the individual difference of the ultrasound observation apparatus.

### Second Embodiment

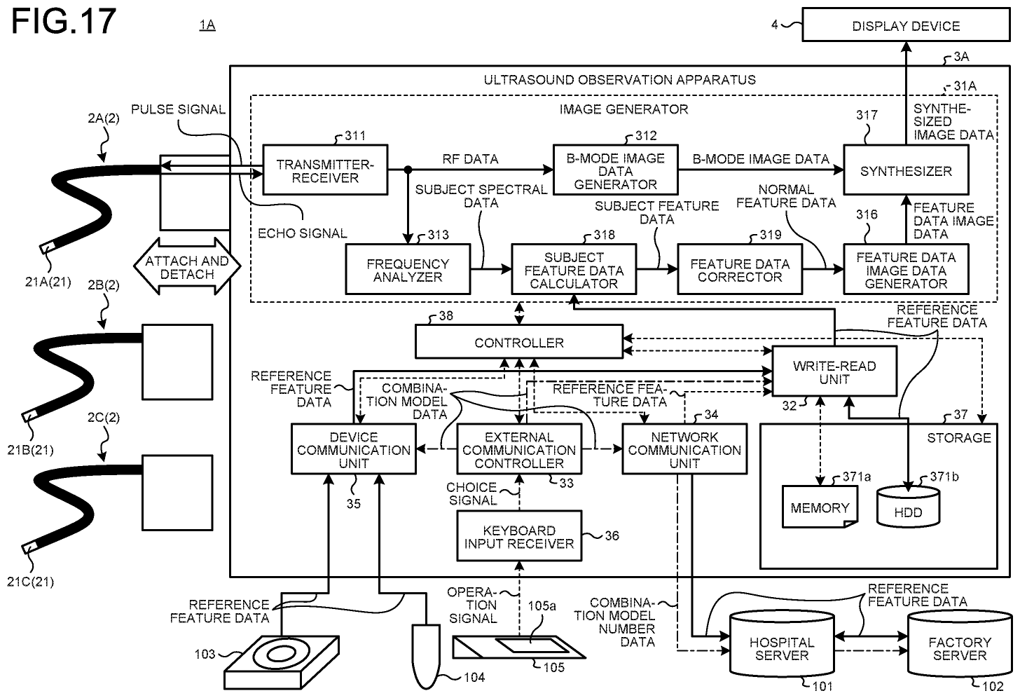

A second embodiment of the disclosure will be described below. FIG. 17 is a block diagram illustrating a configuration of an ultrasound diagnostic system including an ultrasound observation apparatus according to a second embodiment of the disclosure. The first embodiment has been described as one where the spectral corrector 314 corrects the subject spectral data into the normal spectral data and the normal feature data is calculated from the normal spectral data. In the second embodiment, subject feature data is calculated from subject spectral data and, by correcting the subject feature data, normal feature data is calculated.

As opposed to the configuration of the ultrasound diagnostic system 1 according to the above-described first embodiment, an ultrasound diagnostic system 1A according to the second embodiment includes an ultrasound observation apparatus 3A instead of the ultrasound observation apparatus 3. The ultrasound observation apparatus 3A includes an image generator 31A instead of the above-described image generator 31. The ultrasound observation apparatus 3A has the same configuration as that of the above-described ultrasound observation apparatus 3 excluding the image generator 31A.

The image generator 31A includes the transmitter-receiver 311, the B-mode image data generator 312, the frequency analyzer 313, a subject feature data calculator 318 that calculates subject feature data based on subject spectral data that is calculated by the frequency analyzer 313, a feature data corrector 319 that calculates normal feature data by performing correction corresponding to the model and individual of the ultrasound endoscope 2 and the model of the ultrasound observation apparatus 3A on the subject feature data that is calculated by the subject feature data calculator 318, the feature data image data generator 316 that assigns color information according to the normal feature data that is calculated by the feature data corrector 319, and the synthesizer 317 that generates synthesized image data by synthesizing the feature data image that is generated by the feature data image data generator 316 with the B-mode image that is generated by the B-mode image data generator 312.

The subject feature data calculator 318 calculates feature data of subject spectral data (pre-correction feature data) by approximating a plurality of sets of subject spectral data that are output from the frequency analyzer 313 by a straight line and calculates feature data by performing correction of attenuation depending on the frequency on the pre-correction feature data. The feature data calculating method is similar to that of the above-described first embodiment.

The feature data corrector calculates normal feature data by performing correction using the reference feature data on the subject feature data that is calculated by the subject feature data calculator 318. The reference feature data is formed of the reference feature data for model difference correction (corresponding to the first reference data for model difference correction according to the second embodiment) that is obtained by performing regression analysis on the above-described spectral data for model difference correction and reference feature data for individual difference correction (corresponding to the second reference data for individual difference correction according to the second embodiment) obtained by performing regression analysis on the spectral data for individual difference correction. The feature data corrector 319 calculates normal feature data by adding or subtracting the reference feature data for model difference correction and reference feature data for individual difference correction to or from the subject feature data according to Equation (3-1) described above.

The above-described first embodiment demonstrates that, from Equation (3-1), based on the spectral data S(P_{C}A₀; f, z) and S(P₀B₀; f, z), which can be acquired using the reference individuals in a factory, or the like, and spectral data S(PᵢA₀; f, z) that can be acquired using the ultrasound observation apparatus (the ultrasound observation apparatus A₀ herein) that is the reference individual of Reference model A in a factory, or the like, before shipping and each individual Pᵢ of ultrasound endoscope, reference spectral data S(PᵢBₘ; f, z) from combination of a given individual of ultrasound observation apparatus (ultrasound observation apparatus Bₘ) of a model (Model B herein) different from the reference model and a given individual Pᵢ of ultrasound endoscope is calculated. In the second embodiment, it is possible to obtain normal feature data not depending on the model difference and individual difference by correcting subject feature data using reference feature data for model difference correction that is calculated from sets of spectral data S(P₀A₀; f, z) and S(P₀B₀; f, z) and reference feature data for individual difference correction that is calculated from spectral data S(PᵢA₀; f, z). The reference feature data for model difference correction and the reference feature data for individual difference correction are stored previously in the storage 37 or an external storage medium (such as the hospital server 101 or the optical drive 103 described above).

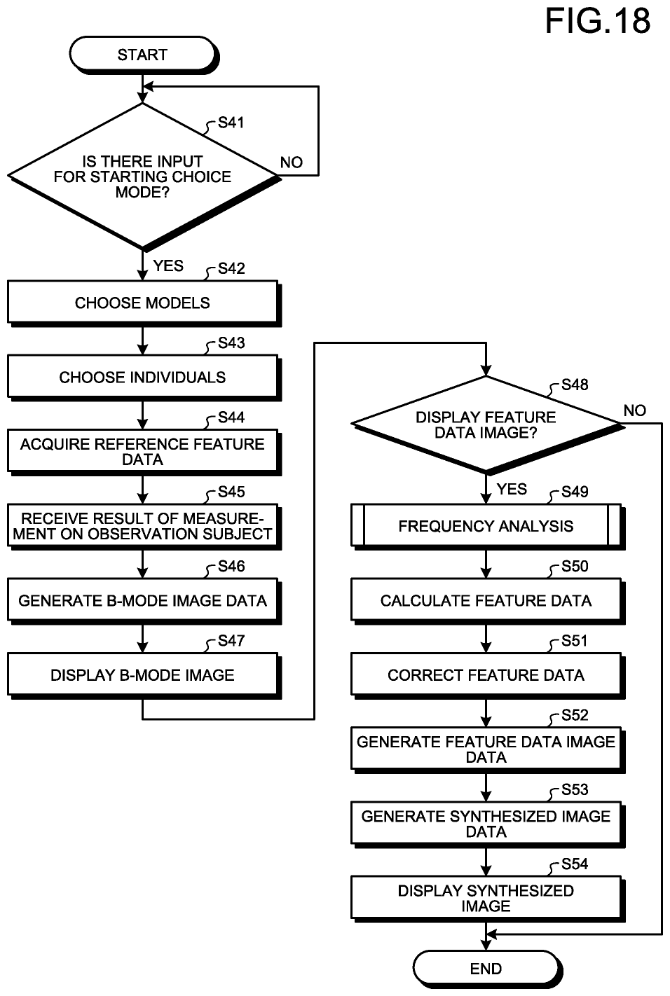

FIG. 18 is a flowchart illustrating an overview of a process performed by the ultrasound observation apparatus 3A having the above-described configuration. First of all, as at step S1 illustrated in FIG. 10 described above, the ultrasound observation apparatus 3A determines whether there is an input of a choice signal for entering a choice mode to acquire reference feature data from the keyboard input receiver 36 (step S41). When there is an input of a choice signal for starting the choice mode to the external communication controller 33 (YES at step S41), the ultrasound observation apparatus 3A moves to step S42. On the other hand, when there is no input of the choice signal for starting the choice mode to the external communication controller 33 (NO at step S41), the ultrasound observation apparatus 3A repeats choice information checking.

At step S42, the external communication controller 33 outputs a read instruction to read a model list and connectability information to the write-read unit 32. The write-read unit 32 searches the storage 37 and reads the model list of the ultrasound observation apparatus 3A, the model list of the ultrasound endoscope 2, and the connectability information on various models that are stored in the storage 37 and outputs the model lists and the connectability information to the external communication controller 33. The external communication controller 33 generates a model choice screen to choose models of the ultrasound observation apparatus 3A and the ultrasound endoscope 2 based on the model lists and the connectability information and causes the touch panel 105a of the keyboard 105 to display the model choice screen via the keyboard input receiver 36.

At step S43, the write-read unit 32 searches the storage 37, generates a list of sets of reference feature data stored in the storage 37 (simply referred to as “reference feature data list” below) and outputs the reference feature data list to the external communication controller 33. In the reference feature data list, the model names and the individual numbers of the ultrasound endoscope 2 and the ultrasound observation apparatus 3A serving as the basis of the sets of reference feature data are associated with the file names of the respective sets of reference feature data. The external communication controller 33 generates an individual choice screen to choose individuals of the ultrasound endoscope 2 based on the reference feature data list and causes the touch panel 105a of the keyboard 105 to display the individual choice screen via the keyboard input receiver 36.

When information on models and individuals is input on the individual choice screen, the external communication controller 33 generates combination model data containing the information on the model of the ultrasound endoscope 2 and the model of the ultrasound observation apparatus 3A and the information on the individuals of the ultrasound endoscope 2 and the model of the ultrasound observation apparatus 3A and outputs the combination model number data to the write-read unit 32.

At step S44, the write-read unit 32 acquires the combination model number data, acquires the reference feature data from the storage 37 or reads the reference feature data by performing control to cause at least any one of the network communication unit 34 and the device communication unit 35 to acquire the reference feature data relating to the chosen models and individuals, and inputs the reference feature data to the spectral corrector 314. When the corresponding reference feature data is not stored in the storage 37, the write-read unit 32 causes at least any one of the network communication unit 34 and the device communication unit 35 to read the reference feature data via the external communication controller 33. The reference feature data acquired herein is, for example, the feature data that is calculated based on the above-described reference spectral data S(PᵢB₀; f, z).

Steps S41 to S44 are executed when the ultrasound observation apparatus 3A is started for the first time or when the choice mode to specify models and individuals is started via the keyboard 105, or the like. When the ultrasound observation apparatus 3A is started for the second time or later or when the choice mode is not started, the ultrasound observation apparatus 3A executes the process of the following steps S45 to S54.

At step S45, the transmitter-receiver 311 receives an echo signal via the ultrasound endoscope 2. The transmitter-receiver 311 amplifies the echo signal. The transmitter-receiver 311 then samples and discretizes the echo signal that is amplified at an appropriate sampling frequency (for example, 50 MHz) to generate RF data and outputs the RF data to the B-mode image data generator 312 and the frequency analyzer 313.

At step S46, the B-mode image data generator 312 amplifies the echo signal, for example, based on the relationship between the amplification factor and the reception depth (STC correction). The B-mode image data generator 312 generates B-mode image data using the RF data after STC correction and outputs the B-mode image data to the synthesizer 317.

At step S47, the synthesizer 317 does not perform any processing on the B-mode image data and directly outputs the B-mode image data to the display device 4. The display device 4 having received the B-mode image data displays the B-mode image corresponding to the B-mode image data.

At step S48, the controller 38 checks which of “displaying” and “not displaying” the feature data image is chosen by the technologist via a button or a menu (not illustrated in the drawings) of the keyboard 105. When choice of “displaying” is confirmed, the controller 38 outputs a feature data image creation start command to each unit forming the image generator 31A (YES at step S48). On the other hand, when choice of “not-displaying” is confirmed, the controller 38 does not output the feature data image creation start command (NO at step S48).

On receiving the feature data image creation start command, the image generator 31A performs the process at and after step S49. Regardless whether there is the feature data image creation start command, the transmitter-receiver 311 and the B-mode image data generator 312 of the ultrasounds observation apparatus 3A repeats the process from step S45 to S47 above. For this reason, while the technologist is issuing an instruction “not to display” the feature data image via the keyboard 105, the B-mode image is displayed on the display device 4 repeatedly each time the observation subject is scanned by the ultrasound transducer 21.

When each unit of the image generator 31A receives the feature data image creation start command, first of all, the frequency analyzer 313 performs frequency analysis on the RF data by FFT operation, thereby calculating spectral data corresponding to all the RF data strings (step S49: frequency analysis step). The frequency analysis is the same process as the process illustrated in FIG. 13.

Following the frequency analysis of step S49, the subject feature data calculator 318 calculates subject feature data using the subject spectral data that is generated by the frequency analyzer 313 (step S50). The subject feature data calculator 318 performs simple linear regression analysis on each of a plurality of sets of subject spectral data corresponding to positions in an analysis area that are generated by the frequency analyzer 313, thereby calculating pre-correction feature data corresponding respectively to the sets of spectral data. Thereafter, the subject feature data calculator 318 performs attenuation correction using the attenuation rate ζ on the pre-correction feature data, which is obtained by performing approximation on each set of spectral data, to calculate feature data after attenuation correction and stores the feature data after attenuation correction in the storage 37. The feature data after attenuation correction serve as subject feature data.

At step S51, the feature data corrector 319 corrects the subject feature data that is calculated by the subject feature data calculator 318, thereby calculating normal feature data. According to Equation (3-1), the feature data corrector 319 corrects the reference feature data by adding or subtracting the reference feature data for model difference correction and the reference feature data for individual difference correction, which are acquired at step S44, to and from the subject feature data, thereby calculating normal feature data.

At step S52, the feature data image data generator 316 generates feature data image data obtained by assigning visual information relative to the normal feature data, which is calculated by the feature data corrector 319, correspondingly to each pixel of the image of the B-mode image data.

At step S53, the synthesizer 317 synthesizes the B-mode image data that is generated by the B-mode image data generator 312 and the feature data image data that is generated by the feature data image data generator 316 to generate synthesized image data that is obtained by superimposing the visual information relative to the feature data onto each pixel of the image of the B-mode image data.

At step S54, under the control of the controller 38, the display device 4 displays a synthesized image corresponding to the synthesized image data that is generated by the synthesizer 317.

In the process series (steps S41 to S54) described above, the process of steps S45 to S47 and the process of steps S49 to S52 may be performed in parallel.

In the second embodiment of the disclosure described above, the subject feature data is calculated from the subject spectral data that is calculated by the frequency analyzer 313 and then the normal feature data is calculated by correcting the subject feature data using the reference feature data calculated from the reference spectral data, which is obtained by imaging the reference specimen. According to the second embodiment of the disclosure, it is possible to perform ultrasound signal correction corresponding to the model difference and individual difference of ultrasound probe and the model difference of the ultrasound observation apparatus 3A.

### Third Embodiment

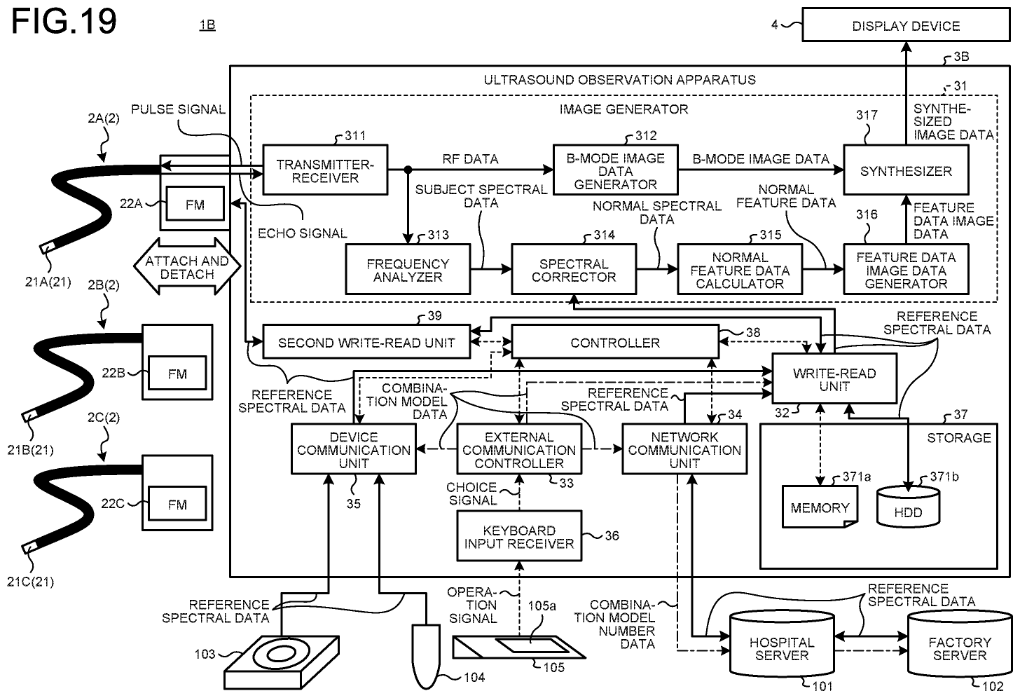

A third embodiment of the disclosure will be described. FIG. 19 is a block diagram illustrating a configuration of an ultrasound diagnostic system including an ultrasound observation apparatus according to the third embodiment of the disclosure. In the third embodiment, the ultrasound endoscope 2 includes a flash memory (FM).

In an ultrasound diagnostic system 1B according to the third embodiment, the ultrasound endoscope 2 includes a flash memory (the ultrasound endoscopes 2A to 2C include a FM 22A, a FM 22B, and an FM 22C, respectively).

As opposed to the configuration of the ultrasound diagnostic system 1 according to the above-described first embodiment, the ultrasound diagnostic system 1B includes an ultrasound observation apparatus 3B instead of the ultrasound observation apparatus 3. As opposed to the configuration of the above-described ultrasound observation apparatus 3, the ultrasound observation apparatus 3B further includes a second write-read unit 39. The ultrasound observation apparatus 3B has the same configuration as that of the above-described ultrasound observation apparatus 3 excluding the second write-read unit 39.

The second write-read unit 39 performs a read process of acquiring, via the write-read unit 32, reference spectral data (including model difference correction spectral data and individual difference correction spectral data described above) that is acquired from any one or both of the network communication unit 34 and the device communication unit 35 and a process of causing the acquired reference spectral data, etc., to be written in the flash memory of the ultrasound endoscope 2.

In the above-described third embodiment of the disclosure, the flash memory (the FM 22A, the FM 22B, and the FM 22C) of the ultrasound endoscope 2 stores the reference spectral data and this allows the ultrasound observation apparatus 3B to which the ultrasound endoscope 2 after storing the reference data is connected to acquire the reference spectral data from the ultrasound endoscope 2. As a result, it is possible to acquire the reference spectral data without the technologist's operation of input to the keyboard 105. According to the third embodiment of the disclosure, it is possible to obtain the effects of the above-described first embodiment and reduce the work of the technologist.

### Fourth Embodiment

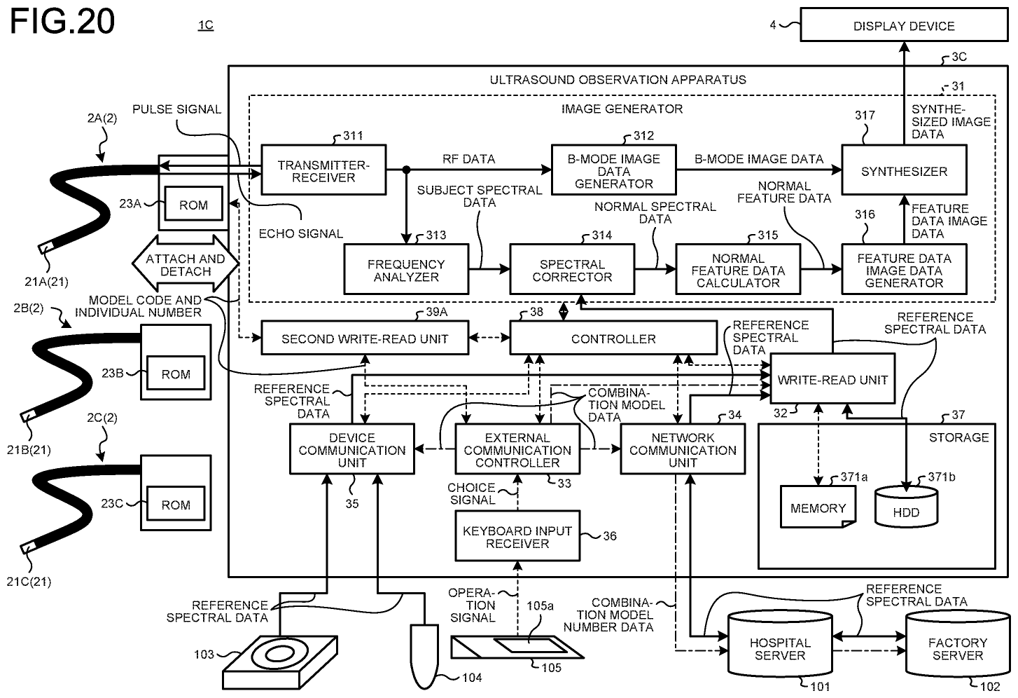

A fourth embodiment of the disclosure will be described. FIG. 20 is a block diagram illustrating a configuration of an ultrasound diagnostic system including an ultrasound observation apparatus according to the fourth embodiment of the disclosure. In the fourth embodiment, the ultrasound endoscope 2 includes a ROM.

In an ultrasound diagnostic system 1C according to the fourth embodiment, the ultrasound endoscope 2 includes a ROM (the ultrasound endoscopes 2A to 2C include a ROM 23A, a ROM 23B, and a ROM 23C, respectively). Each of the ROMs stores a model code representing the model of the ultrasound endoscope 2 and an individual number.

As opposed to the configuration of the ultrasound diagnostic system 1 according to the above-described first embodiment, the ultrasound diagnostic system 1C includes an ultrasound observation apparatus 3C instead of the ultrasound observation apparatus 3. As opposed to the configuration of the above-described ultrasound observation apparatus 3, the ultrasound observation apparatus 3C further includes a second write-read unit 39A. The ultrasound observation apparatus 3C has the same configuration as that of the above-described ultrasound observation apparatus 3 excluding the second write-read unit 39A.

When the ultrasound endoscope 2 is connected, the second write-read unit 39A reads the model code and the individual number from the ROM of the connected ultrasound endoscope 2. The second write-read unit 39A output the read model code to the external communication controller 33.

Based on the model code and the individual number that are input from the second write-read unit 39A and its corresponding model code (of the ultrasound observation apparatus 3C), the external communication controller 33 generates combination model number data in which the models and individuals of the ultrasound endoscope 2 and the ultrasound observation apparatus 3C are associated and outputs the combination model number data to the write-read unit 32. According to a read instruction from the communication unit of the write-read unit 32, the external communication controller 33 chooses the communication unit for connection to acquire the reference spectral data from the network communication unit 34 and the device communication unit 35 and cause the chosen communication unit to read the reference spectral data. The following process is the same as that of steps S5 to S14 of the above-described first embodiment.

In the above-described fourth embodiment of the disclosure, the ROM (the ROM 23A, the ROM 23B, and the ROM 23C) of the ultrasound endoscope 2 stores its corresponding model code and the individual number and this allows the ultrasound observation apparatus 3C to which the ultrasound endoscope 2 is connected to acquire the model code and the individual number from the ultrasound endoscope 2, automatically generate combination model number data based on the model code and the individual number of the connected ultrasound endoscope and its model code, and acquire corresponding reference spectral data. As a result, it is possible to automatically acquire the reference spectral data without an operation of the technologist to make an input to the keyboard 105. According to the third embodiment, it is possible to achieve the effect of the above-described first embodiment and reduce the work of the technologist.

### Fifth Embodiment

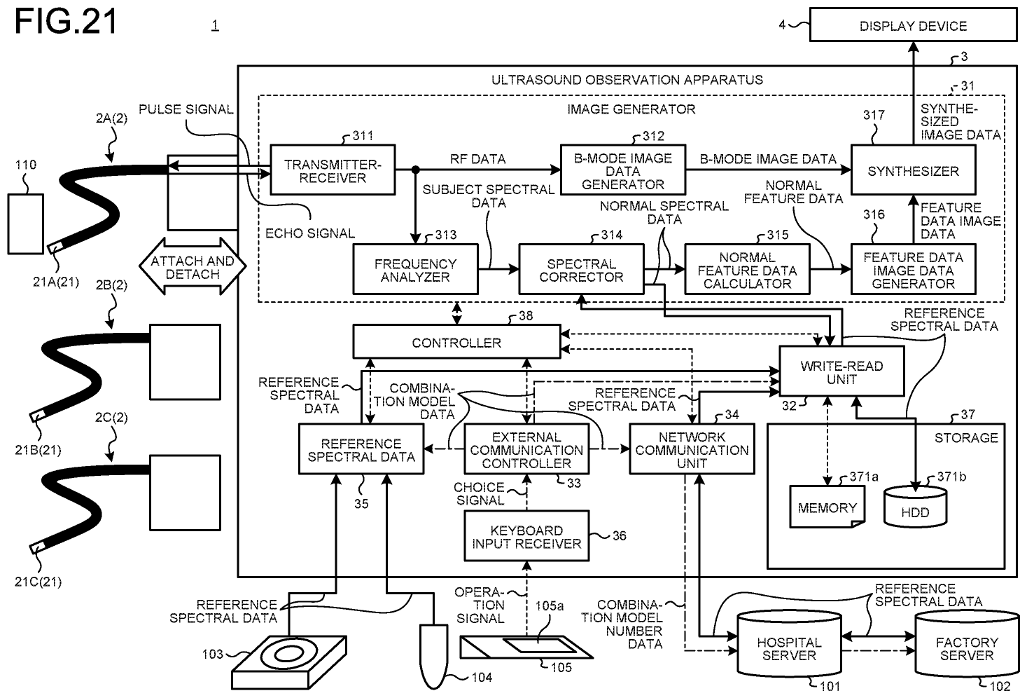

A fifth embodiment of the disclosure will be described. FIG. 21 is a block diagram illustrating a configuration of an ultrasound diagnostic system including an ultrasound observation apparatus according to the fifth embodiment of the disclosure. In the fifth embodiment, the ultrasound endoscope 2 acquires reference spectral data for individual difference correction using a reference specimen 110. The reference specimen 110 used herein is, for example, the same phantom or acryl board as that used for reference spectral data that is acquired previously.

The ultrasound diagnostic system 1 according to the fifth embodiment has the same configuration as that of the first embodiment. Different aspects from those of the first embodiment will be described below.

When the ultrasound endoscope 2 acquires an echo signal from the reference specimen 110, the spectral corrector 314 does not correct subject spectral data that is generated by the frequency analyzer 313 and outputs the subject spectral data to the write-read unit 32 as normal spectral data.

When the normal spectral data is input from the spectral corrector 314, the write-read unit 32 stores the normal spectral data as individual difference correction spectral data in the storage 37. In the storage 37, the individual difference correction spectral data is stored in association with the model and the individual number of the ultrasound endoscope 2. In this manner, for example, in a facility, such as a hospital, it is possible to acquire individual difference correction spectral data. The process performed by the image generator 31 is the same as that of the first embodiment excluding the aspect that the above-described individual difference correction spectral data is stored in the storage 37 in advance.

In the above-described fifth embodiment of the disclosure, the ultrasound endoscope 2 and the reference specimen 110 on the market are used to acquire the individual difference correction spectral data and thus, even when a sensitivity error, or the like, occurs in a facility, such as a hospital, individual difference correction spectral data is acquired using the reference specimen 110 in the facility and the spectral corrector 314 generates normal spectral data using the reference spectral data containing the individual difference correction spectral data, which enables emergency treatment for sensitivity correction.

Embodiments for carrying out the present disclosure have been described; however, the disclosure should not be limited only to the above-described embodiments. For example, in the ultrasound observation apparatus, the circuits having respective functions may be connected via a bus or part of the functions may be incorporated in a circuit structure corresponding to other functions.

In the first to fifth embodiments described above, the reference specimen is described by exemplifying a phantom obtained by uniformly mixing known scatterers whose material, mass density, acoustic velocity, acoustic impedance, diameter and number density are known into a medium whose material, mass density, acoustic velocity, acoustic impedance, diameter and number density are also known. The phantom may be replaced with a subject whose physical amounts, such as a diameter of scatterers, a scattering intensity of the scatterers and a number density of the scatterers, are known and the distribution is uniform. For example, specific tissue, such as the liver of an animal, may be used if physical amounts are known or can be measured accurately. In this case, it is preferable that at least any one of the model difference correction reference data and the individual difference correction reference data be acquired using an echo signal from the reference specimen.

The first to fifth embodiments have been described using, as an ultrasound probe, the ultrasound endoscope 2 including the optical system, such as a light guide; however, the ultrasound probe is not limited to the ultrasound endoscope 2, and an ultrasound probe including no imaging optical system and no imaging device may be used. Furthermore, an ultrasound miniature probe with a small diameter without optical system may be used as the ultrasound probe. The ultrasound miniature probe is generally inserted into a biliary tract, bile duct, a pancreatic duct, a traches, a bronchus, an urethra, an ureter, or the like, and is used to observe the surrounding organs (the pancreas, the lungs, the prostate, the bladder, the lymph node, or the like).

An external ultrasound probe that applies ultrasound from the body surface of an observation subject may be used as the ultrasound probe. The external ultrasound probe is normally made directly contact the body surface when the abdominal organs (the lever, the gallbladder and the bladder), the breasts (particularly, mammary glands), and the thyroid gland are observed.

The ultrasound transducer 21 (the ultrasound transducers 21A to 21C) may include linear transducers, radial transducers, or convex transducers as long as the transducers are of different models. When the ultrasound transducer is a linear transducer, the area scanned by the ultrasound transducer forms a quadrangle (a rectangle or a square) and, when the ultrasound transducer is a radial transducer or a convex transducer, the scanned area forms a sectorial shape or an annular shape. The ultrasound endoscope may cause the ultrasound transducer to perform mechanical scanning or electric scanning that is performed by electrically switching elements that relate to transmission and reception and that are provided in an array as an ultrasound transducer or delaying transmission and reception of the elements.

It has been described that the ultrasound probe and the ultrasound observation apparatus are provided independently. Alternatively, the ultrasound probe and the ultrasound observation apparatus may be configured integrally.

As described above, an operation method of an ultrasound observation apparatus, an ultrasound observation apparatus, and an operation program for an ultrasound observation apparatus according to the disclosure are useful to obtain accurate ultrasound data regardless of a model difference and an individual difference between ultrasound probes and a model difference between ultrasound observation apparatuses.

According to the disclosure, there is an effect that it is possible to obtain accurate ultrasound data regardless of a model difference and an individual difference between ultrasound probes and a model difference between ultrasound observation apparatuses.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An operation method of an ultrasound observation apparatus configured to be able to exchange plural models of ultrasound probes and configured to receive an ultrasound signal from an ultrasound probe connected to the ultrasound observation apparatus, the method comprising:
correcting ultrasound data based on the ultrasound signal using first reference data and second reference data, the first reference data being determined by different models of the ultrasound probes to be connected to the ultrasound observation apparatus, the second reference data being determined by individual probes of the same models of the ultrasound probes to be connected to the ultrasound observation apparatus,
wherein the correcting includes correcting the ultrasound signal by performing a calculation on the ultrasound signal using each of the first reference data and the second reference data for each frequency or each distance.

2. An ultrasound observation apparatus configured to be able to exchange plural models of ultrasound probes and configured to receive an ultrasound signal from an ultrasound probe connected to the ultrasound observation apparatus, the ultrasound observation apparatus comprising:
a corrector configured to correct ultrasound data based on the ultrasound signal using first reference data and second reference data, the first reference data being determined by different models of the ultrasound probes to be connected to the ultrasound observation apparatus, the second reference data being determined by individual probes of the same models of the ultrasound probes to be connected to the ultrasound observation apparatus,
wherein the corrector is configured to correct the ultrasound signal by performing a calculation on the ultrasound signal using each of the first reference data and the second reference data for each frequency or each distance.

3. The ultrasound observation apparatus according to claim 2, wherein at least one of the first reference data and the second reference data is acquired from an echo signal from a reference specimen.

4. The ultrasound observation apparatus according to claim 2, further comprising:
an analyzer configured to analyze the ultrasound signal and calculate spectral data; and
a feature data calculator configured to calculate feature data based on the spectral data that is calculated by the analyzer,
wherein the corrector is configured to correct the spectral data using the first reference data and the second reference data.

5. The ultrasound observation apparatus according to claim 2, further comprising:
an analyzer configured to analyze the ultrasound signal and calculate spectral data; and
a feature data calculator configured to calculate feature data based on the spectral data that is calculated by the analyzer,
wherein the corrector is configured to correct the feature data using the first reference data and the second reference data.

6. The ultrasound observation apparatus according to claim 2, wherein the first reference data is a frequency component of a drive signal to the ultrasound observation apparatus or a different individual of the same model of the ultrasound observation apparatus, a function of frequency of a drive signal to the ultrasound observation apparatus or a different individual of the ultrasound observation apparatus of the same model, or an analysis value based on the frequency component or the function of frequency.

7. The ultrasound observation apparatus according to claim 2, wherein the ultrasound probe includes an ultrasound transducer, and the second reference data is a frequency distribution of sensitivity of the ultrasound transducer, a function of frequency of sensitivity of the ultrasound transducer, or an analysis value based on the frequency distribution or the function of frequency.

8. The ultrasound observation apparatus according to claim 2, further comprising:
an external terminal that is connected to an external device; and
an external communication controller configured to perform control to acquire the first reference data and the second reference data via the external terminal.

9. The ultrasound observation apparatus according to claim 8, further comprising an input receiver configured to receive an input of information on a model and an individual of the ultrasound probe and information on a model of the ultrasound observation apparatus,
wherein the external communication controller is configured to control acquisition of the second reference data on the individual that is specified based on the information received by the input receiver.

10. The ultrasound observation apparatus according to claim 8, further comprising a reader configured to read information for specifying an individual of the ultrasound probe that is connected to the external terminal, from the ultrasound probe,
wherein the external communication controller is configured to control acquisition of the second reference data on the individual that is specified based on the information that is read by the reader.

11. The ultrasound observation apparatus according to claim 8, further comprising a controller configured to perform control to cause the first reference data and the second reference data to be written in a storage medium of the ultrasound probe that is connected to the external terminal.

12. The ultrasound observation apparatus according to claim 2, wherein the corrector is configured to correct the ultrasound frequency signal by adding or subtracting the first reference data and the second reference data to and from the ultrasound signal for each frequency.

13. The ultrasound observation apparatus according to claim 2, wherein the corrector is configured to correct the ultrasound signal by adding or subtracting the first reference data and the second reference data to and from the ultrasound signal for each distance.
